(19) United States
(12) Patent Application Publication
(10) Pub. No.: US 2024/0341620 A1
(43) Pub. Date: Oct. 17, 2024

(54) Wearable Multisensor Patch for Breathing Pattern Recognition

(71) Applicant: University of Windsor, Windsor (CA)

(72) Inventors: Mohammed Jalal AHAMED, Windsor (CA); Simon RONDEAU-GAGNE, LaSalle (CA); Partha Sarati DAS, Windsor (CA)

(21) Appl. No.: 18/625,712

(22) Filed: Apr. 3, 2024

Related U.S. Application Data
(60) Provisional application No. 63/493,829, filed on Apr. 3, 2023.

(51) Int. Cl. A61B 5/08; A61B 5/00; A61B 5/113

(52) U.S. Cl. CPC A61B 5/0816; A61B 5/113; A61B 5/6823; A61B 5/721; A61B 5/725

(57) ABSTRACT

In a preferred embodiment, there is provided a method for determining a breathing pattern, the method comprising: placing a motion sensor and a flex sensor proximate to a diaphragm of a subject; obtaining time domain signals or data from both said sensors over a time period; subjecting the time domain signals or data from the motion sensor to Fast Fourier transformation to obtain frequency domain signals or data, and determining from the frequency domain signals or data one or more frequencies associated with body motion of the subject substantially unrelated to breathing; and filtering the time domain signals or data from the flex sensor to remove information related to the body motion therefrom to obtain filtered time domain signals or data.

The present invention relates to a method and a wearable system for determining a breathing pattern, respiratory events and/or tidal volume, and which preferably operates to filter sensor signals or data to remove or reduce false readings associated with non-breathing-related body movement.

### BACKGROUND OF THE INVENTION

Changes in the rate and depth of breathing are among the earliest vital signals for worsening health and are the most predictive of a concerning clinical outcome. Despite this, easy detection of breathing is missing in most clinical settings and is often left to the nurse for manual recordings. Respiratory rate is in turn a neglected and poorly recorded vital sign. The inaccessibility of an easy respiratory metric, especially over longer durations (e.g., a night's sleep), may prevent early detection of disease progression. Among the different parameters to detect changes in respiration, breathing rate (BR) is particularly relevant, as variations at rest almost always indicate some form of worsening pathology. Despite the fact that BR is a relatively simple phenomenon, its detection can be tedious, and BR measurements can be facilitated by a wearable device that can continuously acquire data and monitor BR without interruption, even during sleep. Recent advancements in the use of wearable sensors for breathing monitoring devices have made it a promising field, as continuous monitoring of a patient's vital signs with wearable technology can enable health professionals to remotely convey medical information from home-monitored patients, allowing them to perform real-time assessments of the patient's physiological status and intervene as needed.

T. Dinh et al., “Stretchable respiration sensors: Advanced designs and multifunctional platforms for wearable physiological monitoring,” Biosens. Bioelectron., 166 (2020): 112460 summarized the latest advancement of stretchable breathing sensors that have been developed using platforms such as textiles and fibers as well as 3D materials such as polymers, nanomaterials, and nanocomposites. T. Dinh et al. “Environmentally friendly carbon nanotube based flexible electronics for noninvasive and wearable healthcare,” J. Mater. Chem. C, 4.42 (2016): 10061-10068 demonstrated the use of a flexible, recyclable, and biodegradable carbon nanotube-based device as a wearble sensitive air flow sensor capable of non-invasively real time monitoring of human breathing. M. Chu et al. “Respiration rate and volume measurements using wearable strain sensors,” npj Digit. Med., 2.1 (2019): 1-9 presented a disposable wearable sensor using piezo-resistive sensors that can measure both the volume and rate of breathing simultaneously and compared it with data obtained from a medical-grade continuous spirometer on participants while they were at rest.

The creation of a respiratory wearable device that is low-cost, low-power, resistant to movement artifacts, and easily accessible on the market is one of the most challenging tasks. Several methods for non-invasive and continuous monitoring of respiration have been proposed. For example, the monitoring of breathing rate and posture movements using pillow and mattress systems was proposed by S. Lokavee et al. “Sensor pillow and bed sheet system: Unconstrained monitoring of respiration rate and posture movements during sleep,” Proceedings of the IEEE International Conference on Systems, Man and Cybernetics, (2012): 1564-1568. The breathing rate may be monitored indirectly using an electrocardiogram (ECG) and a pulse signal. Heart rate (HR) monitoring using a wrist-based accelerometer was proposed by C. Zhao et al. “Robust Heart Rate Monitoring by a Single Wrist-Worn Accelerometer Based on Signal Decomposition,” IEEE Sensor, 21.14 (2021): 15962-15971; however, the downside of this approach is its complexity in application, as well as its sophisticated equipment and limited precision. It is therefore important to reliably monitor the breathing pattern and rate problem. The identification of breathing problems can also be performed with polysomnography (PSG), which is the gold standard for recording cortical, submental muscle, electroocular, body position, ECG, respiration, leg movements, and oxygen desaturation activities during sleep. PSG is, however, large and complex equipment that requires specific lab conditions with specialized staff to be used. Moreover, it is intrinsically unpleasant and can capture only a limited number of hours of sleep. Therefore, PSG cannot be utilized to replicate the regular sleeping data that may be recorded at home over a period of many days. Clinical grade wristband actigraphy, another monitoring device, measures body motion using an accelerometer and calculates sleep/wake states.

H. Scott et al. “A systematic review of the accuracy of sleep wearable devices for estimating sleep onset,” Sleep Medicine Reviews, vol. 49. W.B. Saunders Ltd, February 2020 presented a comprehensive study of the accuracy of wearable devices for sleep monitoring; however, because of a lack of simple algorithms and additional sensors, it often fails to identify immobile wake states because it only has motion information. Therefore, a device that can provide necessary and accurate information on respiration would be highly desirable. There are many wearable sensing systems for sleep and other similar biophysical detection (e.g., wristbands, smartwatches, headbands, rings, smart beds). These sensing devices provide usability, accessibility, and connection but fail to give precise and reliable clinical-graded measurements due to false sensor detection, error and long-term stability. On the commercial market, there are numerous sensor-based wearable devices; however, the majority of them have data acquisition limitations. Moreover, device-to-device output data can lead to confusion. This false detection issue can be resolved by combining two or more sensors, for example, a MEMS accelerometer and pressure sensor, into a single device. For example, C. He et al. “A Smart Flexible Vital Signs and Sleep Monitoring Belt Based on MEMS Triaxial Accelerometer and Pressure Sensor,” IEEE Internet Things J., January 2022 proposed a flexible breath monitoring patch using an accelerometer and pressure sensor. They demonstrated a wearable belt with an accelerometer and a pressure sensor to detect the sleep stages combining body movement and snoring-related vibration events using data from both sensors. They classified different stages of sleep based on the BR, HR, snoring and non-snoring signals.

### SUMMARY OF THE INVENTION

A possible non-limiting object of the present invention is to provide a system or patch system for determining a breathing pattern, breathing rate, breathing types and tidal volume, and which may be manufactured by a less complicated or expensive manner using, for example, low-cost commercially available off-the-shelf (COTS) sensors.

Another possible non-limiting object of the present invention is to provide a system or method for determining a breathing pattern, and which may permit simpler operation to generate breathing data of improved quality, which may be contrasted against the gold standard spirometry data under different movement conditions.

Another possible non-limiting object of the present invention is to provide a system or method for determining a breathing pattern, and which may permit generation of data for use in the recognition of different breathing patterns under different situations, or detection of the onset and peak of breathing activity with different algorithms.

Another possible non-limiting object of the present invention is to provide a system or method for determining a breathing pattern, and which may permit improved wearability for securing breathing data over a longer period of time.

Another possible non-limiting object of the present invention is to provide a system or method for determining a volume of air intake during inhale and exhale estimated from the sensor signal, and which may permit generation of data for use in the recognition of different respirational events under different situations, or detection of the onset and peak of breathing activity.

In one aspect, the present invention provides a device for measuring a breathing pattern, the device comprising an accelerometer for measuring a breathing vibration; a pressure sensor for measuring a breathing pressure; a wearable patch containing the accelerometer and the pressure sensor; and a controller unit for controlling the accelerometer and the pressure sensor, wherein the patch is for placement proximal to the diaphragm.

In one embodiment, the pressure sensor is a capacitive pressure sensor.

In one embodiment, the patch is for placement below the ribs on the upper medial edge of the right lumbar region.

In one embodiment, the device is configured to filter signal data from the accelerometer and the pressure sensor using a type II Chebyshev low-pass filter to obtain noise reduced signal data, preferably wherein the cutoff frequency is 150 Hz and the stop band attenuation is 60 dB.

In one embodiment, the device is configured to apply Hilbert transform to signal data from the accelerometer and the pressure sensor or the noise reduced signal data to obtain analytic signal data.

In one embodiment, the device is configured to apply one or both of breathing onset detection algorithm and breathing peak detection algorithm respectively illustrated by FIGS. 9 and 10.

In yet another aspect, the present invention provides a method for determining a breathing pattern, the method comprising: placing a motion sensor and a flex sensor proximate to a diaphragm of a subject; obtaining time domain signals or data from both said sensors over a time period; subjecting the time domain signals or data from the motion sensor to Fast Fourier transformation to obtain frequency domain signals or data, and determining from the frequency domain signals or data one or more frequencies associated with body motion of the subject substantially unrelated to breathing; and filtering the time domain signals or data from the flex sensor to remove information related to the body motion therefrom to obtain filtered time domain signals or data.

In yet another aspect, the present invention provides a method for determining a breathing pattern, the method comprising: placing a gyroscope sensor and a flex sensor proximate to a diaphragm of a subject; obtaining time domain signals or data from both said sensors over a time period; subjecting the time domain signals or data from the gyroscope sensor to Fast Fourier transformation to obtain frequency domain signals or data, and determining from the frequency domain signals or data one or more frequencies associated with body motion of the subject; and filtering the time domain signals or data from the flex sensor to remove information related to the body motion therefrom to obtain filtered time domain signals or data.

In one embodiment, the one or more frequencies associated with the body motion of the subject at least partially form a peak in the frequency domain signals or data. It is to be appreciated that the motion sensor may provide the time domain signals or data along x-, y- and z-axes. In one embodiment, the time domain signals or data from the motion sensor comprise the time domain signals or data along one or more of x-, y- and z-axes. In one embodiment, the frequency domain signals or data comprise frequency domain signals or data along one or more of x-, y- and z-axes. In one embodiment, the one or more frequencies associated with the body motion of the subject are present in the frequency domain signals or data along one or more of x-, y- and z-axes, or the peak is present in the frequency domain signals or data along one or more of x-, y- and z-axes.

In one embodiment, the breathing pattern comprises a breathing frequency, the method further comprising determining the breathing frequency from the filtered time domain signals or data. It is to be appreciated that the filtering step and the breathing frequency determination step may be performed with the frequency domain signals or data obtained from Fast Fourier transformation of the time domain signals or data from the pressure sensor.

In one embodiment, the breathing pattern comprises a breathing type, the method further comprising: subjecting the filtered time domain signals or data to short-time Fourier transformation with a plurality of shorter time segments over the time period to obtain a spectrogram; identifying a dominant frequency peak for each said segment in the spectrogram; and determining the breathing type for each said segment based on the dominant frequency peak.

In one embodiment, the breathing type comprises slow breathing, normal breathing and fast breathing, the method further comprising determining respective breathing frequency ranges of the slow breathing, normal breathing and fast breathing, wherein said determining the breathing type comprises determining the breathing type for each said segment based on proximity or overlap of the dominant frequency peak to the respective breathing frequency ranges of the slow breathing, normal breathing and fast breathing.

In one embodiment, said determining respective breathing frequency ranges of the slow breathing, normal breathing and fast breathing is performed with a spirometer.

It is to be appreciated that the breathing type may be predefined in other ways that are associated with characteristic breathing frequency ranges of the subject. By way of non-limiting examples, deep and shallow breathing respectively associated with drawing of increased and reduced breath into the lungs may correlate to lower and higher breathing frequency ranges. In one embodiment, the breathing type comprises deep breathing, normal breathing and shallow breathing.

In one embodiment, each said shorter time segment has a duration between about 1 second and about 10 seconds, between about 3 seconds and about 8 seconds, or about 6 seconds, and each said shorter time segment overlaps with at least one other said shorter time segment for an overlap duration between about 0.5 second and about 5 seconds, between about 2 second and about 4 seconds, or about 3 seconds.

In one embodiment, one or both of the motion sensor and the flex sensor are for placement between ribs 7 and 9 on the left side of the subject. In one embodiment, one or both of the motion sensor and the flex sensor are for placement proximate or adjacent to rib 10 on the left side or center of the subject, preferably wherein the breathing type is coughing breathing.

In one embodiment, the method further comprises subjecting the time domain signals or data to one or both of a Savitzky-Golay filter and a low pass filter. In one embodiment, the time domain signals or data from one or both of the motion sensor and the flex sensor are subject to one or both of a Savitzky-Golay filter and a low pass filter after being obtained from the sensor(s) over the time period.

It is to be appreciated that the motion sensor is not particularly limited, provided that the motion sensor detects the body motion unrelated or substantially unrelated to breathing. In one embodiment, the motion sensor comprises an inertial measurement unit having an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data. In one embodiment, the motion sensor comprises one or both of an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data. In one embodiment, the motion sensor comprises an accelerometer. In one embodiment, the motion sensor comprises a gyroscope sensor. In one embodiment, one or both of the accelerometer and the gyroscope sensor are tri-axis sensors.

In one embodiment, the flex sensor comprises a resistive or capacitive flex sensor. In an alternative embodiment, the flex sensor may be replaced by a pressure sensor or a flexible pressure sensor.

It is to be appreciated that the method may be performed with the motion sensor and not the flex sensor, especially if the motion sensor is expected to provide more accurate determination of the breathing pattern, preferably depending on, for example, the breathing type, breathing rate, body motion or type of motion, or others. In one embodiment, the method may be performed with both the motion sensor and the flex sensor, and the breathing pattern may be determined on the basis of which of the motion sensor and the flex sensor are anticipated to provide for more accurate determination.

In yet another aspect, the present invention provides, a patch system for determining a breathing pattern, the system comprising a motion sensor and a flex sensor, both said sensors being placed on a patch for application to skin of a subject between ribs 7 and 9 on the left side of the subject.

In yet another aspect, the present invention provides a system for determining a breathing pattern, the system comprising a motion sensor, a flex sensor and an information processing unit, wherein the sensors are for placement proximate to a diaphragm of a subject, and the information processing unit is configured to: obtain time domain signals or data from both said sensors over a time period; subjecting the time domain signals or data from the motion sensor to Fast Fourier transformation to obtain frequency domain signals or data, and determining from the frequency domain signals or data one or more frequencies associated with body motion of the subject substantially unrelated to breathing; and filtering the time domain signals or data from the flex sensor to remove information related to the body motion therefrom to obtain filtered time domain signals or data.

In yet another aspect, the present invention provides a system for determining a breathing pattern, the system comprising a gyroscope sensor, a flex sensor and an information processing unit, wherein the sensors are for placement proximate to a diaphragm of a subject, and the information processing unit is configured to: obtain time domain signals or data from both said sensors over a time period; subjecting the time domain signals or data from the motion sensor to Fast Fourier transformation to obtain a frequency domain signals or data, and determining from the frequency domain signals or data one or more frequencies associated with body motion of the subject; and filtering the time domain signals or data from the flex sensor to remove information related to the body motion therefrom to obtain filtered time domain signals or data.

In one embodiment, the one or more frequencies associated with the body motion of the subject at least partially form a peak in the frequency domain signals or data.

In one embodiment, the breathing pattern comprises a breathing frequency, the information processing unit being further configured to determine the breathing frequency from the filtered time domain signals or data.

In one embodiment, the breathing pattern comprises a breathing type, the information processing unit being further configured to: subject the filtered time domain signals or data to short-time Fourier transformation with a plurality of shorter time segments over the time period to obtain a spectrogram; identify a dominant frequency peak for each said segment in the spectrogram; and determine the breathing type for each said segment based on the dominant frequency peak.

In one embodiment, the breathing type comprises slow breathing, normal breathing and fast breathing, the information processing unit being configured to determine the breathing type for each said segment based on proximity or overlap of the dominant frequency peak to respective breathing frequency ranges of the slow breathing, normal breathing and fast breathing, wherein the respective breathing frequency ranges of the slow breathing, normal breathing and fast breathing are predetermined from the subject.

In one embodiment, each said shorter time segment has a duration between 1 second and 10 seconds, and each said shorter time segment overlaps with at least one other said shorter time segment for an overlap duration between 0.5 second and 5 seconds.

In one embodiment, one or both of the motion sensor and the flex sensor are for placement between ribs 7 and 9 on the left side of the subject.

In one embodiment, the motion sensor and the flex sensor are for placement on a patch to be applied to skin of the subject.

In one embodiment, the information processing unit is further configured to subject the time domain signals or data to one or both of a Savitzky-Golay filter and a low pass filter.

In one embodiment, the motion sensor comprises an inertial measurement unit having an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

In one embodiment, the motion sensor comprises one or both of an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

In one embodiment, the flex sensor comprises a resistive or capacitive flex sensor.

In one embodiment, the information processing unit is configured to perform the algorithm as illustrated in FIG. 46.

In one embodiment, the system comprises a processing unit configured to receive data wirelessly or wired and process data via an algorithm executed by the processing unit to analyze the data and determine user's respiratory events and provide final values of breathing patterns, type, respiration rate, inhale volume and exhale volume.

In one embodiment, the system comprises a user interface for displaying breathing-related information to the wearer in real-time by wired network and or wirelessly.

In one embodiment, the patch system comprises an inertial measurement unit and a flex sensor, both said sensors being placed on a patch for application to skin of a subject between ribs 7 and 9 on the left side of the subject.

In one embodiment, the system comprises a processing unit for estimating breathing-related tidal volume using a breathing related waveform data from the inertial measurement unit and flex sensor, processing and model fitting the patch output data to estimate tidal volume.

In yet another aspect, the present invention provides a method for determining a respiration events, the method comprising: placing an inertial measurement unit (IMU) positioned on the skin proximate to the diaphragmatic area of the subject to detect breathing-induced acceleration motion and non-breathing related body motion and generate representing time domain data of the signal from the subject; placing a flex pressure sensor positioned on the skin proximate to the diaphragmatic area of the subject to detect breathing-induced muscle stretch and generate representing time domain data of the signal from the subject; obtaining time domain signals or data from both said inertial measurement unit and flex sensors over a time period; subjecting the time domain signals or data from the inertial measurement unit (consisting of tri-axis accelerometer and tri-axis gyroscope) sensor to Fast Fourier transformation to obtain frequency domain signals or data, and determining from the frequency domain signals or data one or more frequencies associated with non-breathing related body motion of the subject; and filtering the time domain signals or data from the pressure sensor to remove information related to the body motion therefrom to obtain filtered time domain signals or data.

In yet another aspect, the present invention provides a system for determining a breathing pattern, the system comprising a gyroscope and accelerometer sensor, a pressure sensor and an information processing unit, wherein the sensors are for placement proximate to a diaphragm of a subject, and the information processing unit is configured to: obtain time domain signals or data from both said sensors over a time period; subjecting the time domain signals or data from the accelerometer sensor to Fast Fourier transformation to obtain frequency domain signals or data, and determining from the frequency domain signals or data one or more frequencies associated with body motion of the subject; and filtering the time domain signals or data from the pressure sensor to remove information related to the body motion therefrom to obtain filtered time domain signals or data.

It is to be appreciated that the breathing pattern may include, but not limited to, breathing rate, breathing type or tidal volume. In one embodiment, the body motion is substantially unrelated to breathing motion. In that embodiment, the method is performed with subjecting the time domain signals or data from the accelerometer to Fast Fourier transformation to obtain the frequency domain signals or data, and the system comprises the accelerometer, the pressure sensor and the information processing unit.

### BRIEF DESCRIPTION OF THE DRAWINGS

Reference may now be had to the following detailed description taken together with the accompanying drawings in which:

FIG. 1 shows a wireless multisensor patch in accordance with a preferred embodiment of the invention;

FIG. 2 shows a photograph of components used for characterization of an accelerometer using the 9100D Portable Vibrometer, and by which three-axis output (X, Y, Z) was obtained and compared according to respective accelerated values;

FIG. 3 shows an accelerometer (LSM9DS1) vibration analysis under different frequencies and acceleration, and which provide for data indicating consistency between different accelerations;

FIG. 4 shows Fast Fourier Transform of the vibration signal seen in FIG. 3 from the accelerometer (LSM9DS1);

FIG. 5 shows a photograph of components used for characterization of a force sensor using an LCR meter;

FIG. 6 shows a graph illustrating response of SingleTact (15 mm Diameter Sensor, 45N/10 lb Force) force sensor under different finger pressures, where capacitance under different finger pressure was obtained, along with tapping frequency as shown in FIG. 7;

FIG. 7 shows a graph illustrating of the force sensor's ability to detect finger tapping conducted at a frequency of 3 Hz, 5 Hz, 8 Hz and 10 Hz;

FIG. 8 shows a graph illustrating spirometer sensor breathing output signal as a reference;

FIG. 9 shows a flow chart of a breathing onset detection algorithm for offline analysis;

FIG. 10 shows a flow chart of a breathing peak detection algorithm for off-line data analysis;

FIG. 11 shows graphs illustrating experimental results of different breathing stages, inhaling, exhaling, holding, and normal breathing, where graph (a) shows accelerometer sensor breathing output signal (9 breaths/min), and graph (b) the pressure sensor breathing output signal (9 breaths/min);

FIG. 12 shows graphs illustrating experimental results of normal breathing, where graph (a) shows those of the accelerometer, graph (b) those of the pressure sensor, and graph (c) those of the spirometer;

FIG. 13 shows a FFT spectrum of the accelerometer and pressure sensor breathing output results from FIG. 12;

FIG. 14 shows graphs illustrating accelerometer sensor and pressure sensor breathing output signal under stand-up and sit-down body movement while sitting, where the colored bar shows the indication of normal breathing, breath holding, and sitting down and standing up motions;

FIG. 15 shows graphs illustrating accelerometer sensor and pressure sensor output signal under left and right body movement while sitting, where the colored bar shows the indication of normal breathing, breath holding, and right-left body movement;

FIG. 16 shows graphs illustrating output signals from the accelerometer and the pressure sensor and the pressure sensor breathing output signal under cough, where the colored bar indicates the missed coughing spikes;

FIG. 17 shows graphs illustrating response time of the accelerometer and the pressure sensor before filtering;

FIG. 18 shows graphs illustrating output signals from the accelerometer and the pressure sensor while sighing, where a missed sighing spike is represented by a colored bar;

FIG. 19 shows graphs illustrating output signals from the pressure and accelerometer sensors while yawning, where the colored bar shows where the yawing spikes were missed;

FIG. 20 shows graphs illustrating output signals from the pressure and accelerometer sensors while deep breathing and walking;

FIG. 21 shows graphs illustrating the accelerometer ((a) and (b)) and pressure sensor ((c) and (d)) breathing onset detection results of slow breathing, fast breathing, and body movement, where the red envelope shows detected breathing activity;

FIG. 22 shows graphs illustrating detected breathing peaks of accelerometer and pressure sensor output signal under slow breathing, fast breathing, and body movement conditions, where the small red circles show detected breathing peaks;

FIG. 23 shows graphs illustrating comparison of the SEM for the breathing pattern of the accelerometer and the pressure sensor for different measurements;

FIG. 24 shows a wired multisensor patch in accordance with a preferred embodiment of the invention;

FIG. 25 shows in (a) an overview of a multisensor data recording system in accordance with a preferred embodiment of the invention; (b) an illustration of a multisensor data process flow diagram for feature extraction and detection; and (c) pictures illustrating inhale and exhale airflow directions;

FIG. 26 shows a drawing illustrating different locations for placement of the patch;

FIG. 27 shows graphs illustrating a 6th-order low-pass Chebyshev Type II filter magnitude and phase responses generated using MATLAB;

FIG. 28 shows a schematic drawing of twelve (12) different locations A1 to A12 selected for a study for placement of the multisensor data recording system, and which cover the chest, diaphragm and stomach areas;

FIG. 29 shows, on the left, an image of an assembled printed circuit board (PCB) showing a preferred embodiment of the multisensor data recording system used in the study referenced in respect of FIG. 28, and on the right, an image of a wireless module;

FIG. 30 shows reference signal from spirometer flow data showing a time domain response showing signal for normal, shallow, yawing, and coughing breathing patterns;

FIG. 31 shows reference signal from spirometer flow data showing a frequency domain response showing signal for normal, shallow, yawing, and coughing breathing patterns;

FIG. 32 shows accelerometer time response showing the signal wave form and strength for all twelve (12) locations when normal breathing was performed (Normal Breathing Time domain signal for Participant-1);

FIG. 33 shows accelerometer time response showing the signal wave form and strength for all twelve (12) locations when shallow breathing was performed;

FIG. 34 shows accelerometer time response showing the signal wave form and strength for all twelve (12) locations when yawning breathing was performed;

FIG. 35 shows accelerometer time response showing the signal wave form and strength for all twelve (12) locations when coughing breathing was performed;

FIG. 36 shows accelerometer response for all twelve (12) locations in the frequency domain for normal breathing pattern;

FIG. 37 shows accelerometer response for all twelve (12) locations in the frequency domain for shallow breathing pattern;

FIG. 38 shows accelerometer response for all twelve (12) locations in the frequency domain for yawning breathing pattern;

FIG. 39 shows accelerometer response for all twelve (12) locations in the frequency domain for Coughing breathing pattern;

FIG. 40 shows a schematic illustrating data acquisition and analysis;

FIG. 41 shows photographs of a breathing sensor, including an IMU and flex sensors;

FIG. 42 shows raw data of accelerometer and flex signals for (a) Normal breathing and (b) shallow breathing;

FIG. 43 shows filtered accelerometer and flex signals for (a) Normal breathing and (b) shallow breathing;

FIG. 44 shows normal Breathing while turning left and right;

FIG. 45 shows holding breath while turning left and right;

FIG. 46 shows a sensor fusion algorithm;

FIG. 47 shows flex sensor data and gyro signals from the IMU sensor during a deep breathing test in the presence of motion;

FIG. 48 shows FFT of the flex sensor signal before filtering (a), FFT of the flex sensor signal after filtering (b), FFT of the flex sensor signal after filtering (b), FFT of the spirometer signal (c) and FFT of the gyro signals of the IMU sensor (d, e, f);

FIG. 49 shows flex sensor data before and after filtering (upper portion) and spirometer data (lower portion) of the deep breathing test in the presence of motion;

FIG. 50 shows a breathing test including deep, normal, and shallow breathing;

FIG. 51 shows STFT of the breathing test including deep, normal, and shallow breathing;

FIG. 52 shows frequency ranges of different breathing types of six persons (3 male and 3 females);

FIG. 53 shows a histogram of the most dominant frequency in each window of time for the breathing test including deep, normal, and shallow breathing;

FIG. 54 shows time domain graphs illustrating determination of a breathing volume from information from a flex sensor under normal breathing; and

FIG. 55 shows time domain graphs illustrating determination of a breathing volume from information from a flex sensor under deep breathing.

### DETAILED DESCRIPTION OF THE PREFERRED EMBODIMENTS

In a preferred embodiment, a wearable and flexible patch using acceleration and pressure is provided. The multisensory patch may be integrated into a flexible platform with die scale bonding that can make it less bulky and comfortable to wear. Flexible and stretchable breathing sensors based on printed circuit boards and other devices may be adapted to create multifunctional and multimodal platforms with higher sensitivity.

In a preferred embodiment, breathing-related skin and muscle movement in thoracic and abdominal areas may be captured by using a wearable sensor provided with an accelerometer and a pressure sensor. A multisensor patch may aid in the identification of breathing patterns. Two distinct sensors may be employed, an accelerometer to detect breathing vibrations and a pressure sensor to measure breathing pressure, and combining the data may permit more consistent, accurate and dependable results. Data from both sensors under different movements may be used to identify and correct false detection of motion that is not related to breathing. Such approach and related devices to collect breathing pattern data and comparing these results simultaneously may permit more accurate information about breathing patterns, which may not be obtained with an accelerometer or a pressure sensor alone. This information may permit improved use in diagnosing respiratory health conditions.

It has been appreciated that the wearable multisensor patch may provide a more complete picture of the breathing pattern. The accelerometer and the pressure sensor signal may show different quantitative features, which may be used to classify and detect different breathing patterns. The sensors may be used to more precisely measure various human breathing circumstances. It has been envisioned that the combination of the sensors in the patch may provide for more reliable detection of breathing signals, in circumstances where, for example, one sensor cannot detect a breathing event under mechanical disturbances, and the other sensor may assist in the recognition of the unnoticed breathing event.

### System Description and Methodology

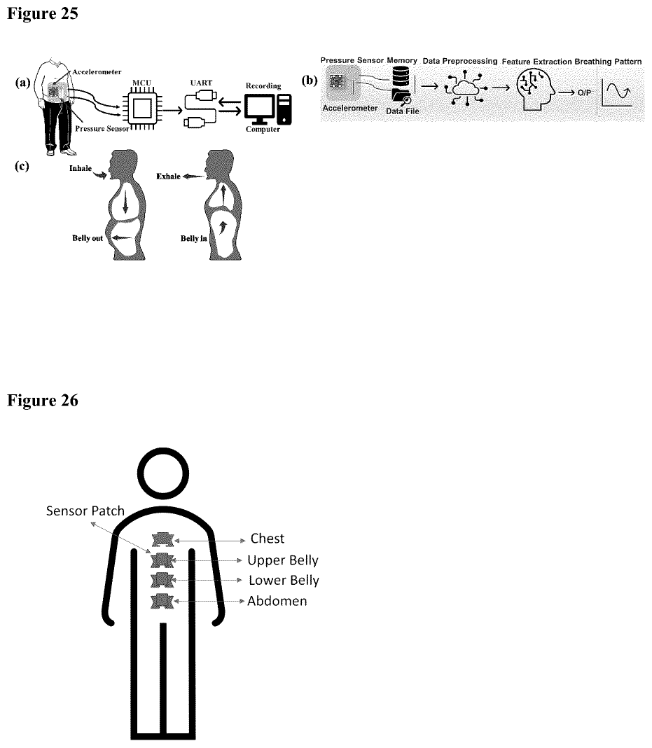

As seen in FIG. 25, a human breathing monitoring system or multisensory patch was implemented based on a noninvasive measurement method. As seen in FIG. 25(a), the system mainly includes three subsystems: a sensor subsystem, a microcontroller subsystem, and a recorder subsystem. FIG. 25(b) shows a data flow chart of breathing sign detection. During inhalation and exhalation, there are periodic volume changes in the thoracic and abdominal areas with movements on the surface. These changes could be detected by accelerometers, gyroscopes, pressure sensors and various sensors based on different mechanisms. As seen in FIG. 25(c), during inhalation, the contraction of the diaphragm pushes the abdominal organs downward and causes the intrathoracic pressure to fall, and during exhalation, the diaphragm relaxes, the chest and abdomen return to the rest position, and lung volumes diminish.

### The Accelerometer

To accurately monitor BR, an accelerometer was first used to track respiratory motion. The breathing signal can be obtained when the accelerometer is properly positioned over the diaphragm. During breathing, the position of the accelerometer relies on chest expansion and contraction. Three linear acceleration readings can be obtained in orthogonal directions through a triaxial accelerometer. An accelerometer block diagram is shown in FIGS. 25(a) and(b). The system used COTS to make a multisensor patch. The SparkFun 9-DoF IMU Breakout-LSM9DS1 was used as the accelerometer for breathing measurement. Table 1 below summarizes the characteristics of the accelerometer.

TABLE 1
Performance summary of the accelerometer

| Characteristics | Value | |
|---|---|---|
| Linear acceleration full scale | ±2/±4/±8/±16 | g |
| Acceleration Channels | 3 | |
| Sensor mass | 0.23 | grams |
| Supply Voltage | 3.3 | V |
| Sampling Frequency | 100 | Hz |
| Number of Bits | 16 | bits |

| | |
|---|---|
| Serial Interface | I2C(100 kHz) |

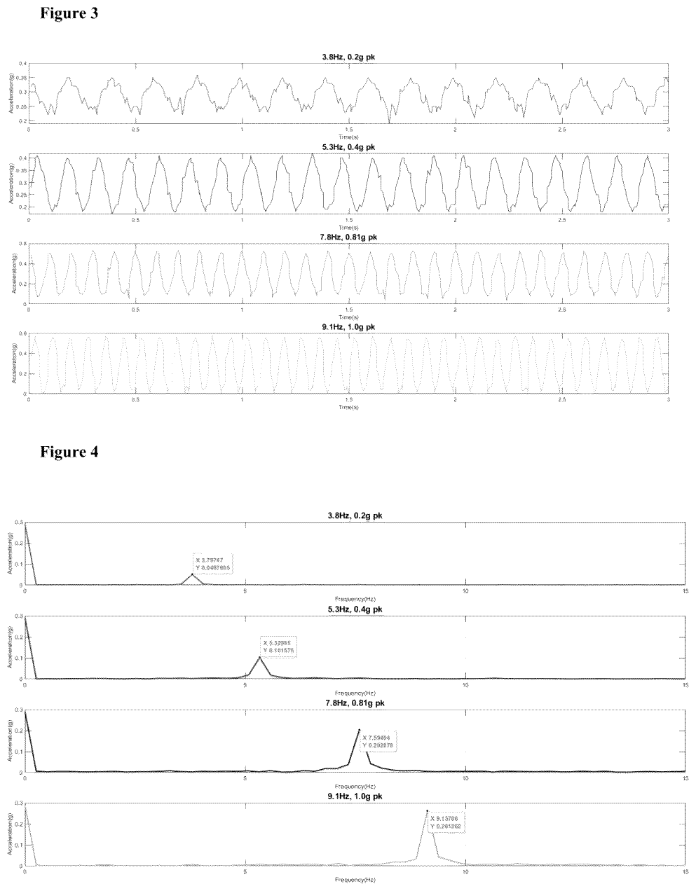

FIG. 2 shows an experimental setup for a vibration analysis of the accelerometer using a 9100D Portable Vibrometer. FIG. 3 shows that the accelerometer may acquire different accelerations under different frequencies, which was verified by Fast Fourier Transformation (FFT), as seen in FIG. 4. The minimum vibration frequency that can be analyzed using the 9100D was 5 Hz, showing that the accelerometer is compatible with the breathing application.

### Pressure Sensor

Capacitive pressure sensors detect changes in electrical capacitance induced by diaphragm movement to determine pressure. A capacitive pressure sensor was selected for different breathing pressure measurements, in view of high sensitivity and frequency response, as well as lower hysteresis value and better repeatability. A SingleTact (PPS UK Limited, Diameter=15 mm, Force=45 N/10 lb) capacitive force sensor was used as the pressure sensor to mitigate possible accelerometer problems while breathing, as seen in FIGS. 25(a) and(b). Arduino UNO is a microcontroller board based on the ATmega328P. Characteristics of the pressure sensor are summarized in Table 2 provided below.

TABLE 2
Performance summary of the pressure sensor

| Characteristics | Value | |
|---|---|---|
| Sensor Diameter | 15 | mm |
| Sensor mass | 0.23 | grams |
| Force Resolution | <0.2% of Full-Scale Range (FSR) | |
| Maximum Force | 300% of FSR | |
| Linearity Error | <2% | |
| Hysteresis | <4% | |
| Sensor Response Time | <1 | ms |
| Contact Surface Material | Polyimide | |
| Full Scale Range | 90 g-45 kg (100 lbs) | |
| Minimal Detectable Force | 0.882N | |
| Supply Voltage | 3.7 | V |
| Sampling Frequency | 60 | Hz |
| Number of Bits | 10 | bits |

| | |
|---|---|
| Serial Interface | I2C(100 kHz) |

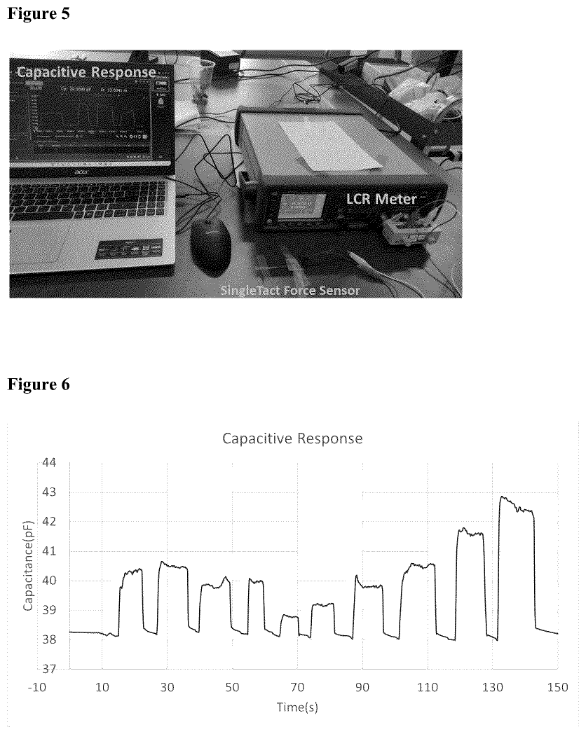

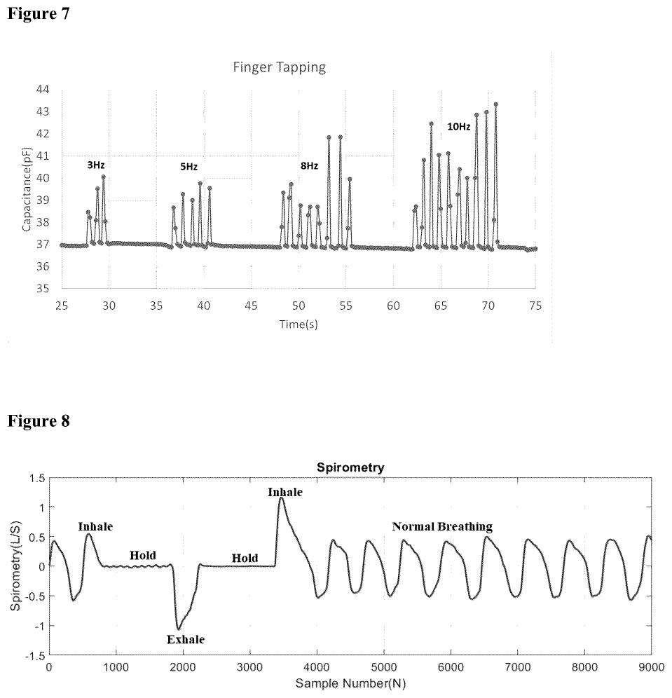

FIG. 5 shows an experimental setup for the capacitive force sensor analysis using the Precision LCR Meter (E4980AL) from 20 Hz to 300 kHz. FIG. 6 shows that the capacitive force sensor could detect different small-scale forces effectively, making it suitable for use with the path. FIG. 7 shows finger tapping output results under different frequencies.

### Data Collection and Processing

The pressure sensor and the accelerometer were fixed on the skin immediately below the ribs on the upper medial edge of the right lumbar region. A long wire was used to connect the microcontroller and a recording laptop. The device was instrumented on four co-authors for analysis. In the initial subset of breathing maneuvers, the pressure sensor and accelerometer were contrasted against gold-standard spirometry. For the spirometry setup, tidal volume and rate were measured by breathing through a mouthpiece connected to a respiratory flow head and high precision spirometer (ADInstruments®, Colorado Springs, USA). Respiratory rate and volume were collected in real time using PowerLab and LabChart Pro software (ADInstruments®).

As seen in FIG. 8, several recording sessions were performed corresponding to different breathing conditions, consisting of quiet breathing, breath holds at functional residual capacity, and breath holds at total lung capacity. All breathing maneuvers were performed in the supine position. Another set of experiments was performed with the pressure sensor and accelerometer only, consisting of 1) five simulated yawns; 2) five simulated coughs; and 3) five simulated sighs. A serial terminal was employed to record the data from the accelerometer and pressure sensor data acquisition software to store data from the pressure sensor.

### Onset Detection of Breathing Patterns

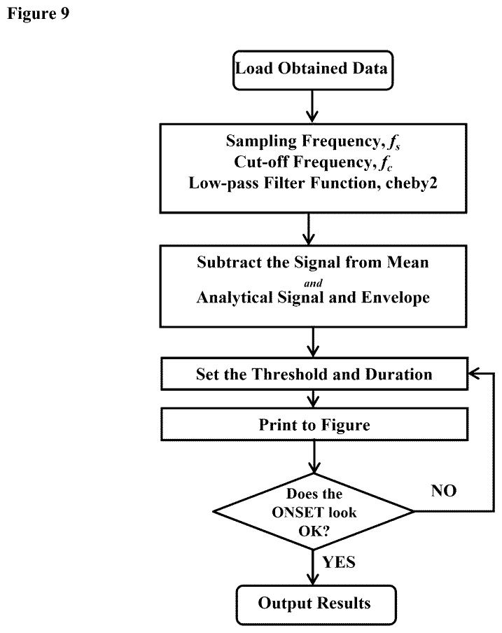

Once the corresponding acceleration and pressure sensor signal was acquired, the envelope and detailed signals were obtained using Hilbert transformation. The onset detection flow chart is shown in FIG. 9. A real function x(t) of the Hilbert transform xH(t) is defined as:

$$x H ( t ) = \frac{1}{π} {\int }_{- ∞}^{+ ∞} x ( τ ) \frac{1}{t - τ} d τ = x ( t ) * \frac{1}{π t} . \qquad ( 1 )$$

According to Equation (1), H(t) is generated by passing the signal x(t) through a linear time-invariant filter with an impulse response equal to (πt)⁻¹. Using the Fourier transform yields the following results:

$$\begin{matrix}F x H ( t ) = - j sgn ( F x ( t ) ) , & ( 2 ) \\ where & \end{matrix}$$

$$sgn ( . ) = \{ \begin{matrix}+ 1 , & iff ( . ) > 0 , \\ 0 , & iff ( . ) = 0 , \\ - 1 , & iff ( . ) < 0 .\end{matrix} \qquad ( 3 )$$

Since the amplitude of F (x(t)) remains constant, all frequency components shifted by −90° using the Hilbert transform. The function x(t) and transformed xH(t) generate an analytic signal that can be expressed as:

$$z ( t ) = x ( t ) + jxH ( t ) . \qquad ( 4 )$$

Then, the envelope of z (t) is:

$$B ( t ) = \sqrt{{x}^{2} ( t ) + {x}_{H}^{2} ( t )} . \qquad ( 5 )$$

### Peak Detection Analysis

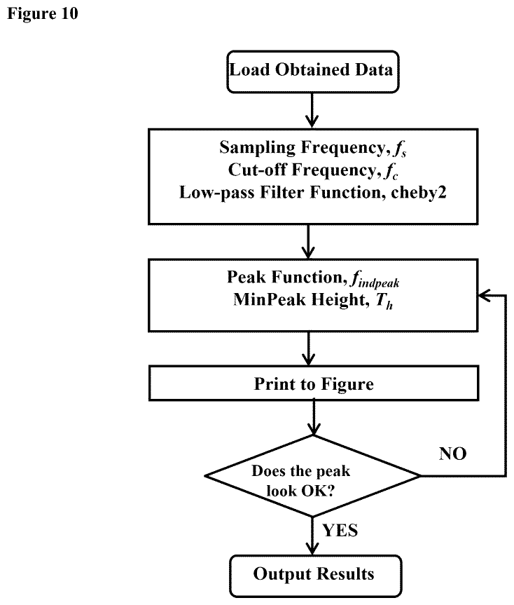

MATLAB (MathWorks, R2021a) was used to process and analyze the data. FIG. 10 shows a flow chart of the standard peak detection. The ‘findpeaks’ function in MATLAB was applied to carry out a standard peak detection algorithm. The ‘findpeaks’ function was given as findpeaks (data, samplingfrequency, ‘MinPeakHeight’,0.2), where ‘MinPeakHeight’ represents the threshold of the minimum amplitude detected at the peak.

Efforts have been made to analyze various respiration signals and develop a method to comprehend the breathing pattern. The sampled accelerometer data contained relatively high-frequency noise as well as an offset due to gravity. The signals acquired by the accelerometers and the pressure sensor were therefore filtered and detrended to remove these components. A type-II Chebyshev low-pass filter (LPF) was used to conduct the extractions. The cutoff frequency was 150 Hz, and the stop band attenuation was 60 dB. The signals were filtered to avoid phase distortions. The same preprocessing steps were applied to other waveforms to ensure synchronization with the accelerometer and pressure sensor data in terms of breathing spikes. The Z-axis acceleration signal was used to analyze breathing patterns. The transfer function of the LPF is described as:

$$H ( z ) = \frac{B ( z )}{A ( z )} = \frac{b ( 1 ) + b ( 2 ) {z}^{- 1} + … + b ( n + 1 ) {z}^{- n}}{1 + a ( 2 ) {z}^{- 1} + … + a ( n + 1 ) {z}^{- n}} \qquad ( 6 )$$

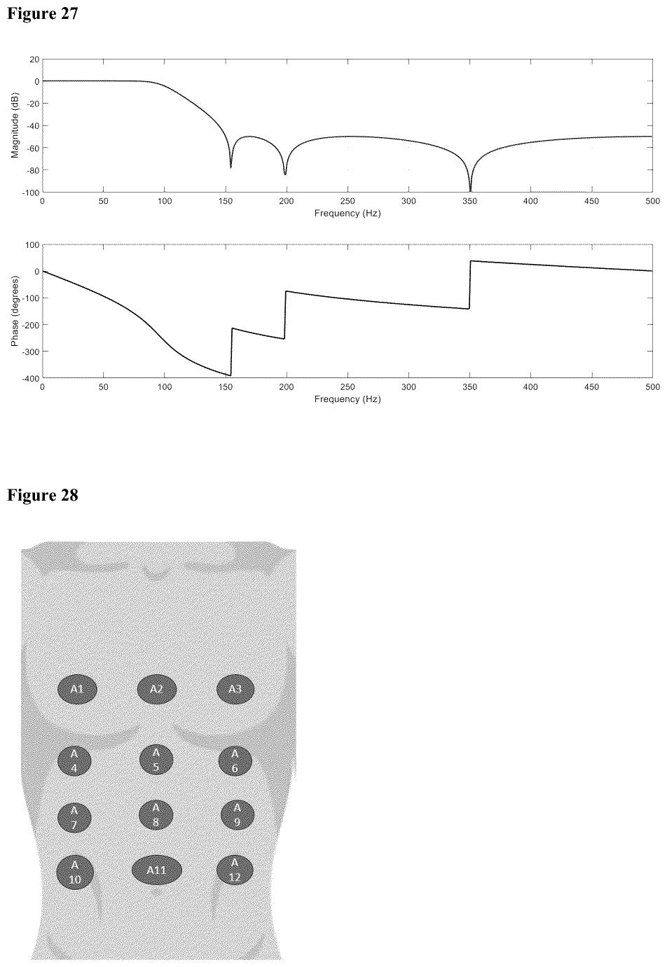

The magnitude response is given by

$$H ( j Ω ) = \frac{ϵ {C}_{N} ( {Ω}_{2} / Ω )}{A ( z )} \qquad ( 7 )$$

where ∈ is a constant, Ω is the 3 dB cutoff bandwidth and CN is a polynomial function. The magnitude response has a maximally flat pass band and equiripple stop band. Here, the magnitude and phase response of the LPF are illustrated, as seen in FIG. 27.

The SingleTact graphical user interface (GUI) was used as a recording tool for pressure sensor data acquisition. The GUI was scaled from 0 to 511, and the measured force is given by equation (9).

$$Output = \frac{C {P}_{r} - C {P}_{b}}{D S V} + 255 \qquad ( 8 )$$

$$Force = \frac{Output / SR}{512} N \qquad ( 9 )$$

$$Pressure = \frac{Force}{SA} \qquad ( 10 )$$

where CPᵣ is the raw capacitance, CP_{b} is the baseline capacitance, DSV is the digital scaling value, SR is the sensor rating, and SA is the sensor area.

### Results and Discussions

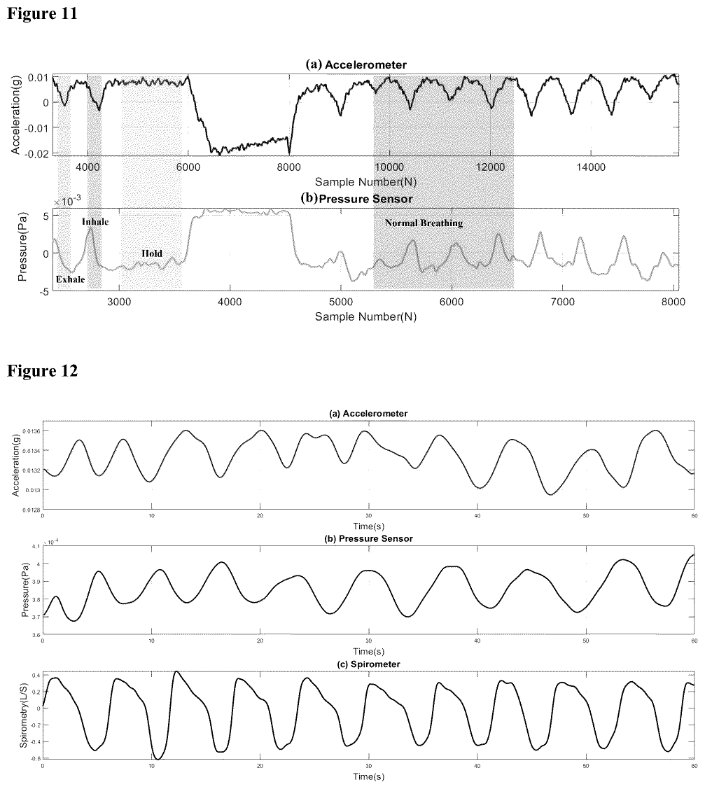

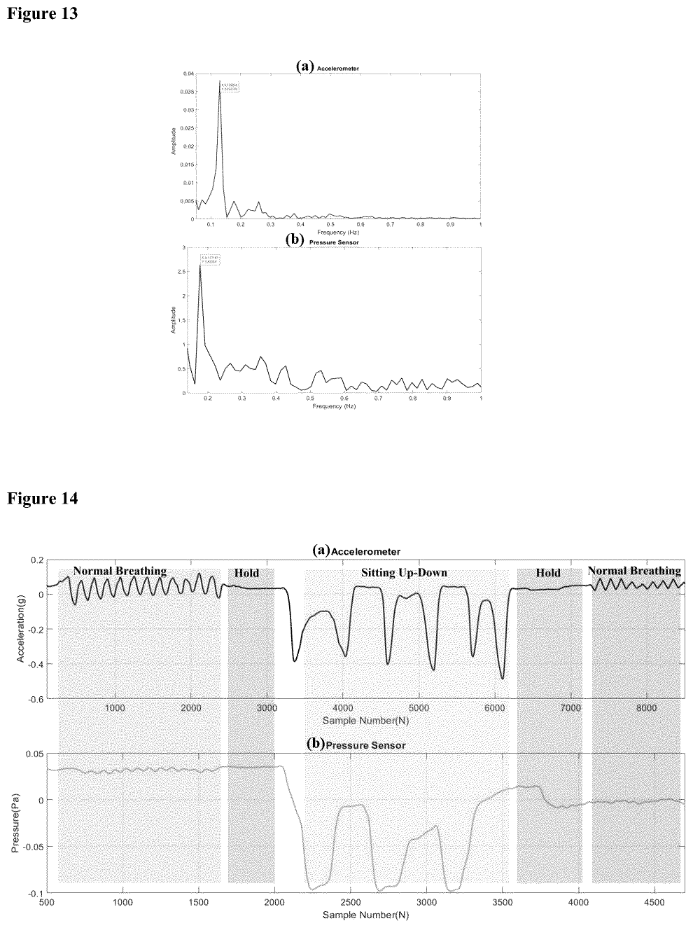

The patch has been envisioned for use in identifying and detecting breathing patterns based on accelerometer and pressure sensor outcomes. A series of breathing experiments were performed. Before starting the experiments, the accelerometer and pressure sensor were placed immediately below the ribs on the upper medial edge of the right lumbar region. Spirometry data were used as a reference. FIG. 11 depict the accelerometer and pressure sensor output signals, which clearly show the aligned holding, inhalation, exhalation and normal breathing patterns. The accelerometer and pressure sensor correctly depicted the various stages of the breathing pattern compared to spirometry, as seen in FIGS. 8 and 11. FIG. 12 shows the visual comparison of normal breathing of the multisensor patch and the gold standard spirometer. The breathing pattern can be easily detected, but it is difficult to identify all the patterns clearly when the human body is in movement. FIG. 13 shows the FFT results of the obtained accelerometer and pressure sensor.

Breathing with Stand-up and Sit-down Motion

Breathing pattern detection during movement is difficult due to the fluctuation of the sensor. For that reason, the multisensor patch is useful in order to recognize the right pattern. Various experiments were performed under human body movement conditions. FIG. 14 shows the experimental results of breathing with stand-up and sit-down conditions. In this situation, oscillations in pressure and acceleration may cause noise artifacts like body movements which can generate wrong results. The accelerometer and pressure sensor output values caused by external noise sources were found to have a major influence on the results, as shown in FIG. 14. During the stand-up and sit-down protocols, the pressure sensor had an accurate indication compared to the accelerometer.

Breathing with Left-side and Right-side Body Movement

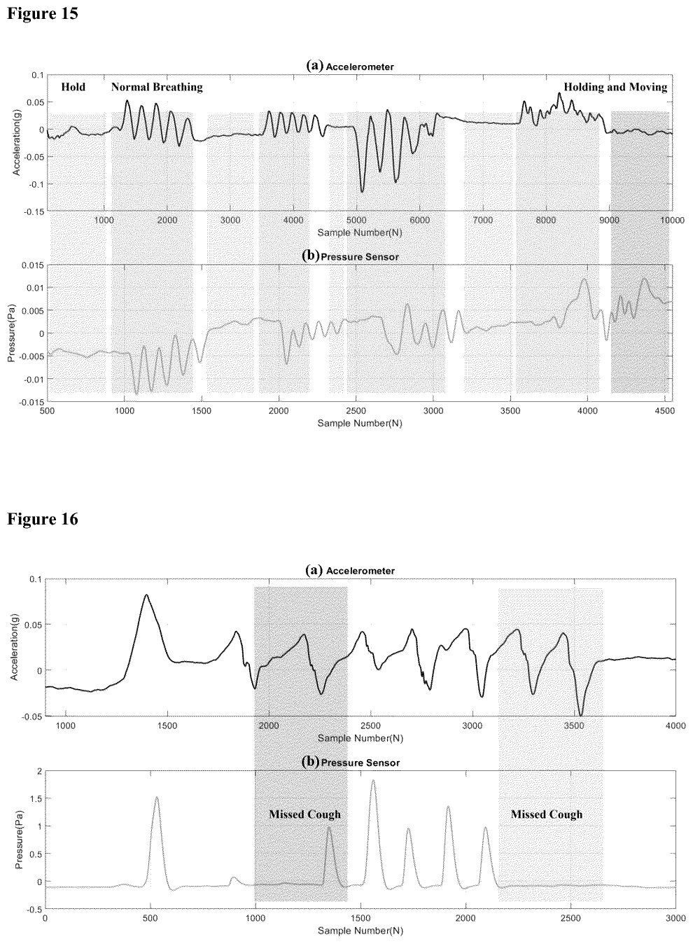

The accelerometer and pressure sensor used in this experiment were able to distinguish between breathing activity and other types of body movement that occur in response to external disturbances. To verify the situation, breathing pattern was obtained while the human body was in movement. In this experiment, the accelerometer showed no acceleration changes even while the body was moving, but the pressure sensor sensed movement despite the absence of breathing. FIG. 15 shows accelerometer and pressure sensor output results where the human body is in the left-right moving condition. The subject was asked to sit on a chair and hold his breath while moving the upper portion of the body left side and then the right side. As indicated in FIG. 15, breathing was held for a specific period in the end. That moment was detected clearly using an accelerometer. The accelerometer signal level was flat, which is correct, but the pressure sensor gave some indication of a signal (spikes), which is similar to the previous normal breathing pattern but occurs due to body movement. The accelerometer and pressure sensor value should not change while a person holds their breath.

### Coughing

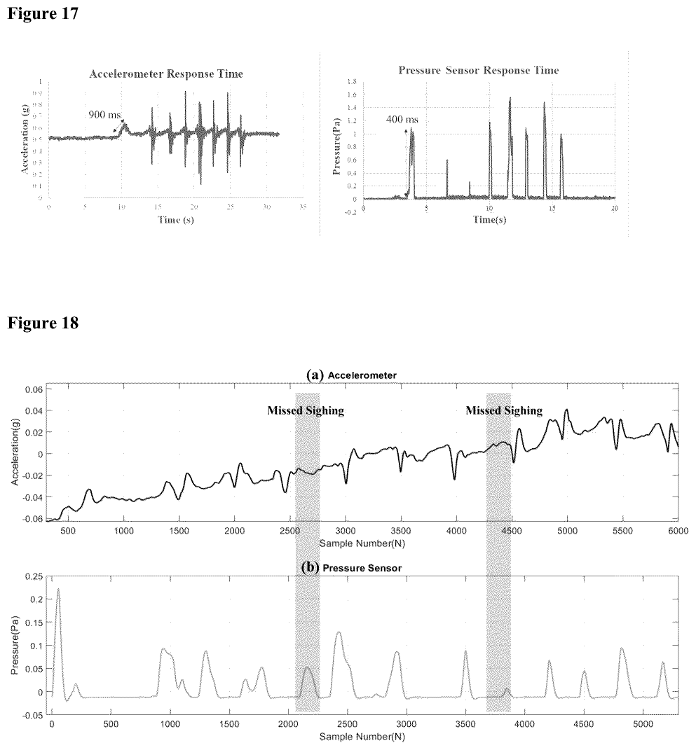

A common sign of many diseases is coughing. As seen in FIG. 16, within a single recording epoch, simulated coughing events were repeated several times. The pressure sensor and accelerometer showed specific waveforms during a cough. In FIG. 16, typical cough waveforms were displayed. Wave height is a measure of cough intensity on a large or small scale. The ability of the pressure sensor and accelerometer to detect coughing events was confirmed, but the pressure sensor missed some cough events (indicated by color marking), whereas the accelerometer detected those missed events. The pressure sensor picked up seven peaks after eight coughs, but the accelerometer picked up eight. The response times of the accelerometer and pressure sensor were 900 ms and 400 ms, respectively, as shown in FIG. 17. Even though the response time of the pressure sensor was lower than that of the accelerometer, since there was an air gap between the two conductive layers, the pressure sensor may occasionally not function.

### Sighing

Similar to breathing, sighing also occurs unintentionally. A session was recorded while simulating sighing. The accelerometer was unable to accurately detect every sighing event, as seen in FIG. 18. The pressure sensor was successful in identifying missed sighing events. There were a total of 13 sighing events, but the accelerometer missed two of them, which are indicated by colored bars. It is understandable that if the intensity of the breathing activity is not strong enough, it will be difficult for both sensors to detect that event effectively, even though the pressure sensor detected those events. One of the event wave height amplitudes was low, despite the pressure sensor's detection of those events.

### Yawning

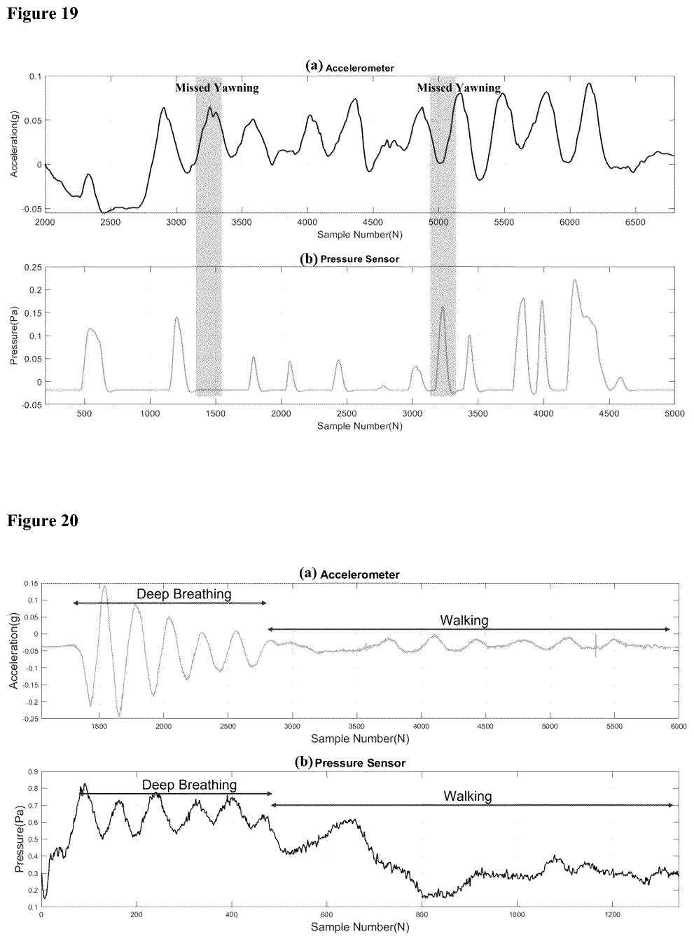

As seen in FIG. 19, yawning breathing issues were tested and confirmed because being overly tired or exhausted is a common reason for excessive yawning. The simulated yawning events were played back several times within a single recording. The pressure sensor effectively detected the yawning event when the accelerometer had missed it, as seen in FIG. 19. The pressure sensor missed one yawning event, but the accelerometer successfully picked it up. Therefore, it is confirmed that the pressure sensor and accelerometer complement one another and improve the precision of breathing signal pattern detection.

### Deep Breathing and Walking

As seen in FIG. 20, deep breathing and walking patterns were tested and verified because detecting breath patterns under mechanical disturbances is very difficult. Within a single recording, the breathing events were repeated numerous times. FIG. 20 shows that the pressure sensor failed to pick up the breathing event while walking, but the accelerometer successfully recognized most of them. The accelerometer also missed some breathing events. In that situation, the pressure sensor detected the unnoticed pattern. Thus, it was confirmed that the pressure sensor and accelerometer may operate cooperatively and increase the accuracy of detecting breathing signal patterns. To improve the accuracy of breathing pattern detection under mechanical disturbances, flexible and stretchable electronics along with artificial intelligence may be used.

### Onset and Peak Detection

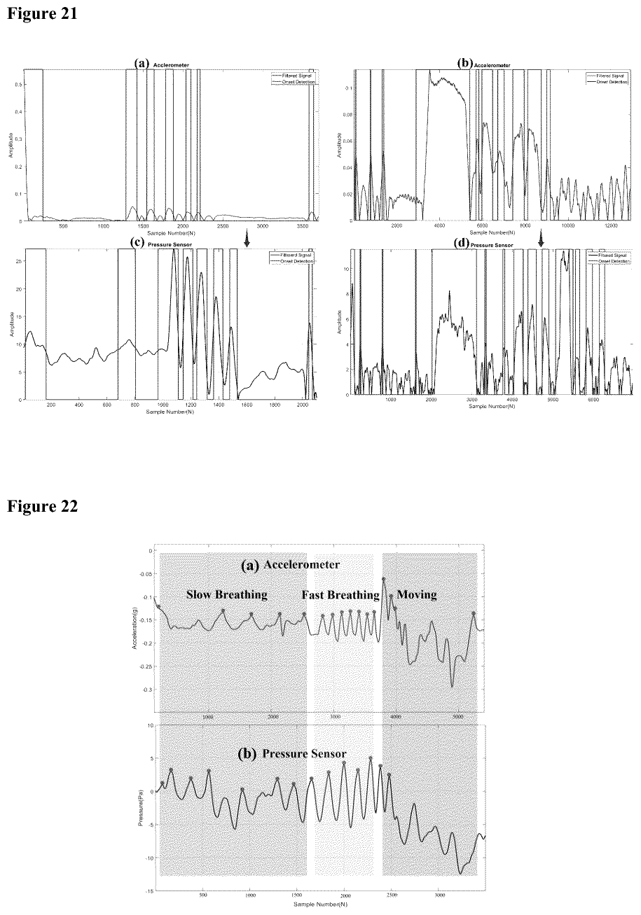

The onset and peak detection algorithm as described in FIGS. 9 and 10 were applied. The threshold and duration of the signal were applied to obtain the onset activity. FIG. 21 shows the two different breathing activities of onset detection from the obtained accelerometer and pressure sensor signals. The detected onset can be easily identified in both the accelerometer and pressure sensor signals. The peak detection results were obtained, and it can be seen in FIG. 22 that the peak detection algorithm identified correct peaks. When the subject was in motion, the peak detection algorithm could not detect the breathing peak because of the threshold problem.

### Standard Error of the Mean

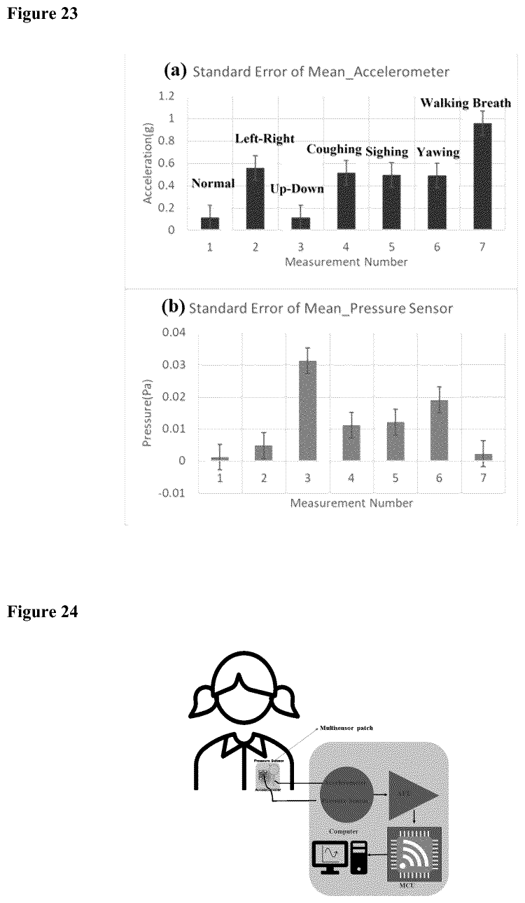

The standard error of the mean of the accelerometer and pressure sensor output were calculated and the results were plotted, as seen in FIG. 23. The standard errors of the mean (SEM) quantify how much the average accelerometer or pressure sensor data are expected to deviate from the real sample mean. To obtain the standard error of the mean, the standard deviation was divided by the square root of the number of samples (accelerometer or pressure data). The SEM can be expressed as:

$$SEM = \frac{σ}{\sqrt{n}} \qquad ( 11 )$$

where σ is the standard deviation and n is the number of samples. As seen in FIG. 23, the length of an error bar and histogram revealed that the accelerometer and the pressure data have significant error variation on the walking breath, left-right movement breath, and updown movement breath. Breathing detection of movement is difficult to identify because of external disturbances. Hence, two sensors were used to reduce error and detect right breathing activity. To detect breathing patterns while being subjected to various mechanical disturbances, the sensor patch may be held firmly in place. The pattern can change, and motion artifacts can be generated due to changes in the orientation of the accelerometer and the air gap difference of the pressure sensor caused by movement of the subject, disturbance of the sensor patch, friction, and slipping. A soft, flexible sensor may stretch, make a strong contact, and maintain it even when moving. It can also slide and rub against the skin without creating motion artifacts. Even if there is movement, only a small portion of the sensor may move, and the overall contact area may not significantly changes, so as to reduce motion artifact.

### Conclusions

Growing demand for compact, highly accurate devices that can measure multiple vital signs is a result of rising health consciousness. The multisensor patch may permit recording of abdominal pressure and acceleration, as well as distinguishing between various breathing patterns that are clinically significant. The multisensor patch may more rapidly identify breathing events linked to sleep apnea, potentially allowing less expensive and more practical alternatives to conventional PSG for noninvasive sleep monitoring at home and in the clinic. A technique for tracking breathing patterns with two sensors is described with experiments to demonstrate practical application in home or real-world situations. The multisensor patch permitted increased accuracy where one sensor was unable to detect a breathing event and the other sensor was able. In cases where yawning breathing occurred, the sensors operated cooperatively to detect events. It has been appreciated that the patch may permit respiration detection in a range of ambulatory settings. The multisensor patch may permit use for monitoring patients with chronic respiratory conditions on a daily basis.

In a preferred embodiment, the patch is a wired multisensor patch, which is a human motion measurement system, having an accelerometer and a pressure sensor connected to an analog front-end unit, a microcontroller unit, and a data logger unit. All sensors and devices were powered and controlled throughout by a microcontroller and data logger module. A general overview of the system is illustrated in FIG. 24. Arduino UNO was used. The board has 6 analog input pins.

It contained a USB port that can be used as a serial communication port with a computer to transfer data. The Arduino graphical user interface was used to control the sensor and a data logger to get the data to get time information obtained from different sensors. The I2C protocol was used. Accelerometer and pressure sensor data were delivered via the Arduino USB connection and stored in a text file on a laptop using the opensource software CoolTermWin.

In a preferred embodiment, there is provided a lightweight, error-free, wearable human motion detection system for integration into the body and clothes for long-term signal acquisition. A low-cost, Bluetooth-based wearable device for human motion detection was made. The device was based on a multisensory patch with an Arduino LilyPad (ATmega328V) microcontroller module and the two sensors, or namely, an accelerometer and a pressure sensor. Arduino LilyPad is specially designed for wearable applications. All the analog sensors, or the accelerometer and the pressure Sensor, were small and lightweight, which can be easily placed on the body.

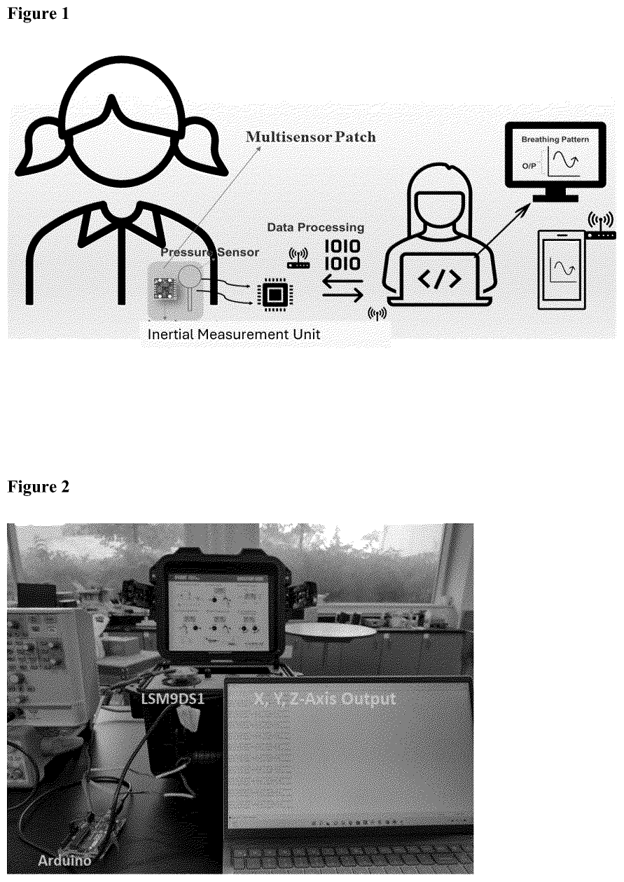

FIG. 1 shows the wireless multisensor patch. The patch was placed on the human chest to detect human motion. A Bluetooth module (HC-08) was used to transfer and receive two analog signals. A single rechargeable lithium battery was used. A mobile application (Human motion detection app) was designed to provide a user-friendly interface for receiving and displaying transmitted data in real-time. Analog data was obtained using analog front-end circuitry. Distinct signal patterns were detected and analyzed by different algorithms. To investigate signal abnormal patterns from complex datasets, the obtained signals were analyzed offline using different algorithms in order to provide accurate human motion detection. The algorithms may handle a vast amount of complex data coming from two different sensors. Arduino-based interfacing software was used to utilize the data and interpret them in order to decide whether breathing or other human motion is happening.

The wearable multisensor detection system may permit a technique to realize desired breathing or other human motion by optimizing size as well as using different multimodal signals rather than using bulky equipment and conventional sensor. The patch included a signal conditioning unit, power delivery unit, data processing, and wireless transmission to a smartphone android app unit or a computer. The patch was fully integrated and miniaturized because wearable devices are subject to the stress of regular human wear and physical exercise.

It has been envisioned that the patch may be placed anywhere around the diaphragm. Experiments or studies may be conducted to identify ideal placement locations with maximum and minimum signal strengths, and/or a self-calibration mechanism may be utilized by users for placement purposes around the lunge, while permitting use of all features. The self-calibration mechanism preferably measures signal strength and set control and tuning parameters. The multisensor patch may be placed in four different places: the upper part of the belly, the lower part of the belly, chest and abdomen as indicated in FIG. 26, and which may permit acquisition of breathing signal of improved quality.

It has been recognized that during inhalation, the contraction of the diaphragm pushes the abdominal organs downward and causes the intrathoracic pressure to fall, and during exhalation, the diaphragm relaxes, the chest and abdomen return to the rest position, and lung volumes diminish. The user may adjust patch location based on, for example, user experience and/or an algorithm dedicated to breathing rate detection, pattern recognition, and signal analysis. The algorithm may be used to remove noise and improve signal quality, or compare different locations data to determine best location for improved sensitivity. Statistical analysis may also be performed to assess different breathing scenarios for different demographics, physical construction, and aged participants.

### Sensor Placement

In a separate study, a wearable inertial accelerometer sensor device was tested in various locations on the chest and diaphragmatic area and on the belly to investigate preferred locations for breathing detection. Twelve (12) different locations and different participant data were analyzed, covering breathing patterns, such as normal breathing, shallow breathing, yawning, and coughing on the test participant, with a view to determine preferred location or area to mount an inertial measurement unit (IMU) accelerometer. It has been recognized that pattern monitoring may be an important indicator of different health or physiological situations. For example, Yawning can reflect on the tiredness level of the person and one of its uses may reside in tracking alertness in drivers. Physiological benefit of yawing may include increasing blood flow to the skull and helps to cool down the facial temperature.

For this study, various methods were used to determine preferred locations based on performance of a microcontroller-based data acquisition system. Both reference signal and signals at different locations were measured for fixed time following same test pattern. One key criteria for performance was similarity of the signals with the reference signal, and the locations with breathing pattern resembling the reference signal were chosen. The resemblance was based on frequency components of the signal. Another criterion in determining preferred locations was the muscle movement and presence of the main inspiratory muscle and intercostal muscles, as well as the diaphragm. The muscle movement involves synchronous movement of upper and lower part of thorax with the abdomen. The middle thoracic sector lies between ribs fifth and twelfth. In this study, the movement and quality of signal were studied particularly in this region for multiple healthy young participants to verify that the location works for all.

### Methodology and Setup

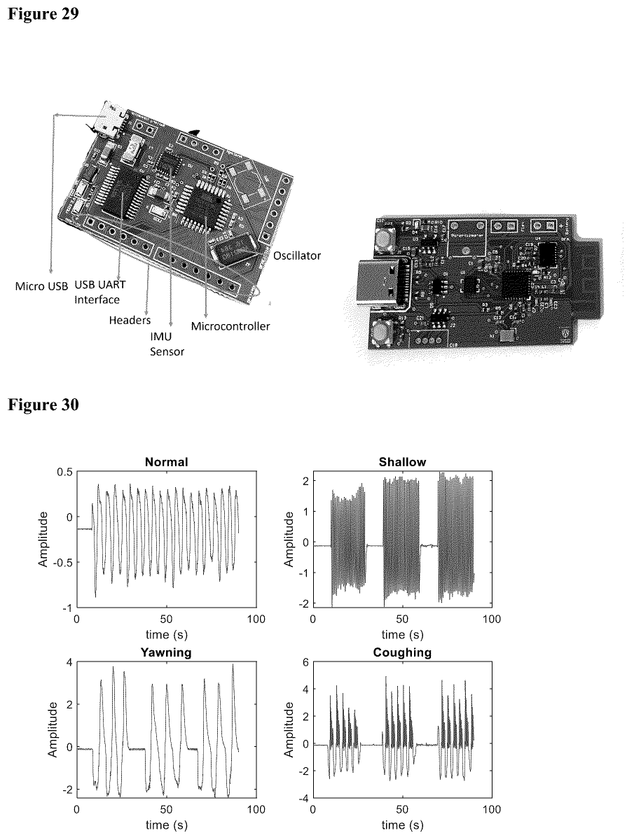

As seen in FIG. 28, twelve (12) test locations were chosen and then narrowed down to four (4) different locations for the reasons based on the muscle movements and sorted results from the first participant. Diaphragm being the main inspiratory muscle, contacts ribs 7-12 (costal part). The external intercostal muscles help the ribs to expand, and internal intercostal muscle pull in the ribs hence help to contract. The locations A5, A6, A8 and A9 are present in the area between ribs 7-12. In this study, as shown in FIG. 29, a microcontroller-based data standalone system was developed. This board was custom designed for the purpose specifically to measure the breathing and includes an inertial sensor, or namely, an IMU. The entire board was connected to a USB B port of a laptop using USB micro-USB type A cable. An Atmega328P microcontroller with a clock speed of 20 MHz was used as the brain of a data acquisition system. The board was programmed using Arduino IDE and Serial port data read was done using MATLAB, with real time visualization of the z axis of the accelerometer. IMU is 3-axis accelerometer, which is low powered and capable of sensing small motion like motion of the chest, abdominal area and was used in this study. The test time was set as 90 seconds and the total number of samples collected are 1654 in 90 seconds. For all the test, the initial 10 seconds started as holding the breath. For normal breathing and yawning, the rest of the remaining 80 seconds the participant breathed normally. Shallow breathing and coughing were kept to 15-20 seconds, depending on the comfort of the participant.

The participants were young healthy males and healthy females all with no medical history for lung related issues. The test location covered the locations of the chest and muscle involved while breathing. Wide area was chosen to determine preferred location to mount the IMU. The IMU covered in a 3D printed case with thin base material was mounted on the skin and attached with a tagederm pad. The test subject was lying on the bed with belly up for all the test cases in this study. The test subjects were also asked to do all breathing exercises with no other body movement except for the breathing patterns. This study was done in Human Kinetics breathing testing lab, University of Windsor.

### Results and Discussions

### A. Reference Signal

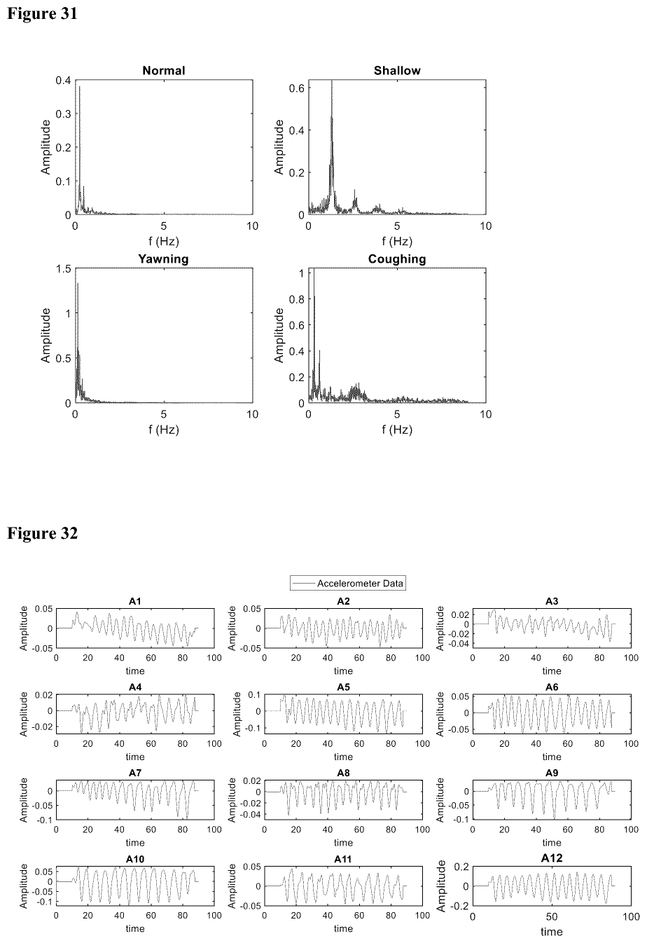

For comparison, spirometer data was used as the reference signal (see FIGS. 30 and 31). This is a medical instrument which has the capability to measure breathing patterns with high precision and accuracy. With sampling rate of 1000, this device is commercially used in medical industry and highly reliable. Two software used were Power Lab and Lab Chart Pro Software (ADI Instrument) to collect data in real time for normal breathing, shallow breathing yawning and coughing of the participant. The spirometer comes with a mouthpiece and nose clip to be worn by the participant while performing various breathing exercises.

Removal of gravitational offset was done from each dataset for each location. The mean of the value was taken and then subtracted before doing any further study or using the data collected. From the collected data, unwanted noise and spikes were removed using a filter like Savitzky-Golay filter and low pass filter with pass band frequency of 0.4 and stop band frequency of 0.8, since the breathing rate do not exceed more than 60 beats per minute the parameters where optimum for this use. Frequency for normal breathing is 0.23 Hz, that for shallow breathing 1.29 Hz, yawning 0.13 Hz and coughing 0.29 Hz.

### B. Time Domain Comparison

For time domain analysis of the signal, the data collected for normal breathing, shallow breathing, yawning, and coughing from each location were compared to the reference signal in time domain. In the case of normal breathing and yawning, the number of breath cycle is compared to that for the reference signal. In the case of shallow breathing and coughing, it was checked whether the number of coughing peak and shallow peak are the same or not.

Breathing rates can be determined by doing peak count. To find the peak count in the time domain data, MATLAB's predefined function was used, which on giving minimum difference between the two cycles and time duration of the breathing experiment returns the peak of breathing signal.

The comparison was done for all the locations on the chest as shown on FIG. 28. Savitzky-Golay filter was chosen to keep the filtered signal very close to the raw data signal, hence not affecting amplitude much. The Savitzky-Golay filter is a digital filter which uses least square estimation and is good in smoothening out the raw data. The parameters like order and frame length were chosen such that it is good enough for the signal while keeping the most prominent features of the signals intact and only smoothening out the spikes in the signal. The initial and last 10 seconds data was not considered for comparison, so as to avoid variation due to processing delays of the electronic system.

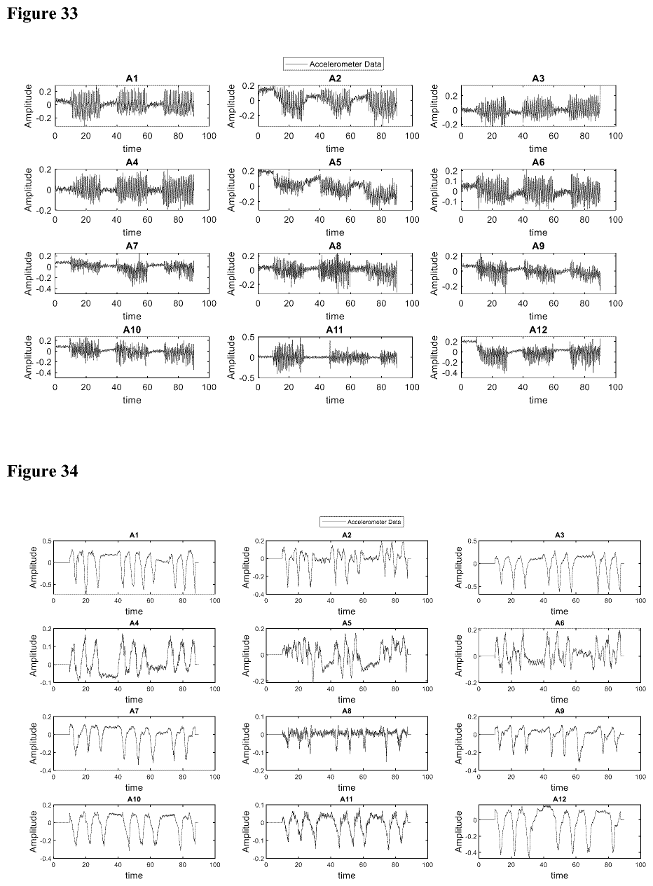

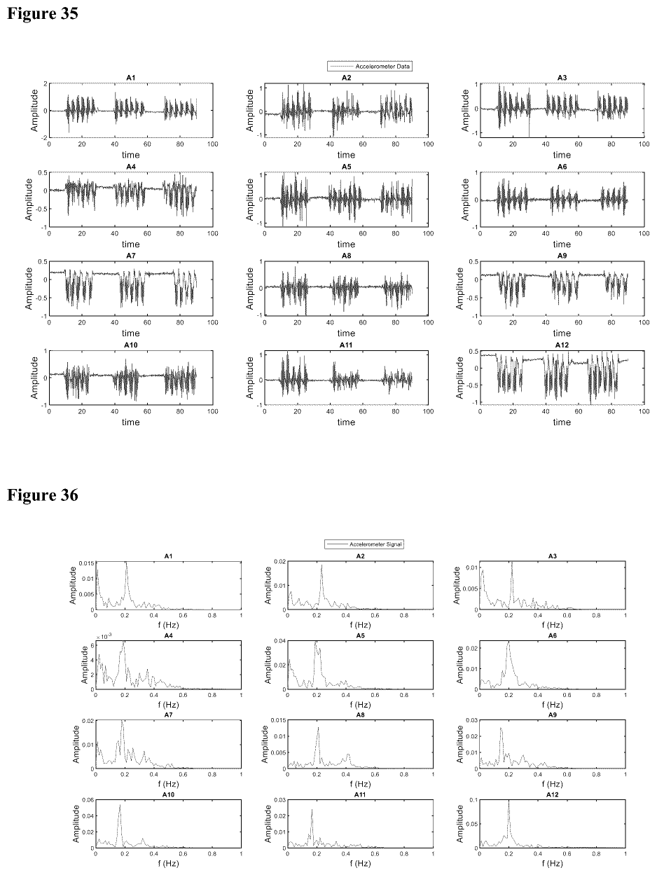

As seen in FIG. 32, for normal breathing patterns, the locations A6, A9, A10, A12 retained most of the characteristics of the signal like the reference signal. For shallow breathing, as seen in FIGS. 33, A1, A4, A6 and A8 were preferable and for yawning A4 and A6 (see FIG. 34). For coughing (see FIG. 35), it was more difficult to determine by visual comparison, hence the results were quantized on frequency domain. However, it appeared that all locations were at least able to detect different breathing patterns. The breathing sensor was able to detect movement of the chest for different breathing patterns.

### C. Frequency Based Analysis

The time domain data above showed an inertial sensor placed in the diaphragmatic area may permit capturing of valuable amplitude and time information of breathing patterns and dynamics. By analyzing the frequency content of these signals, specific breathing patterns, breathing periods, irregularities, and respiratory events such as coughing, yawing or sighs may be identified. Analysis of frequency contents and amplitude may permit more accurate characterization of the breathing.

### Normal Breathing

Breathing frequency, which measures the number of breaths in one second, may be important in monitoring the health of an individual. After signal processing, the z-axis signal coming from twelve (12) different locations were analysed in the frequency domain. To obtain the frequency domain data, MATLAB's predefined function was used with proper sampling rate and the result was plotted against the frequency. For all the different locations, the plots are shown in FIG. 36. As seen in FIG. 36, location A1 showed two prominent frequencies at 0.01 Hz and 0.21 Hz. The higher 0.47 Hz was due to the fact the distance between signal crest was smaller, as clearly seen in FIG. 36. Also, the distance between signal was irregular, and therefore, multiple frequencies were obtained. The locations A12 having 0.20 Hz, A11 having frequency 0.17 Hz, A10 having frequency 0.17 Hz, A8 having frequency 0.21 Hz, and A6 with frequency 0.20 Hz were preferable. In short, the locations A6, A5, A8, A8, A10, A11 and A12 were preferable.

### Shallow Breathing

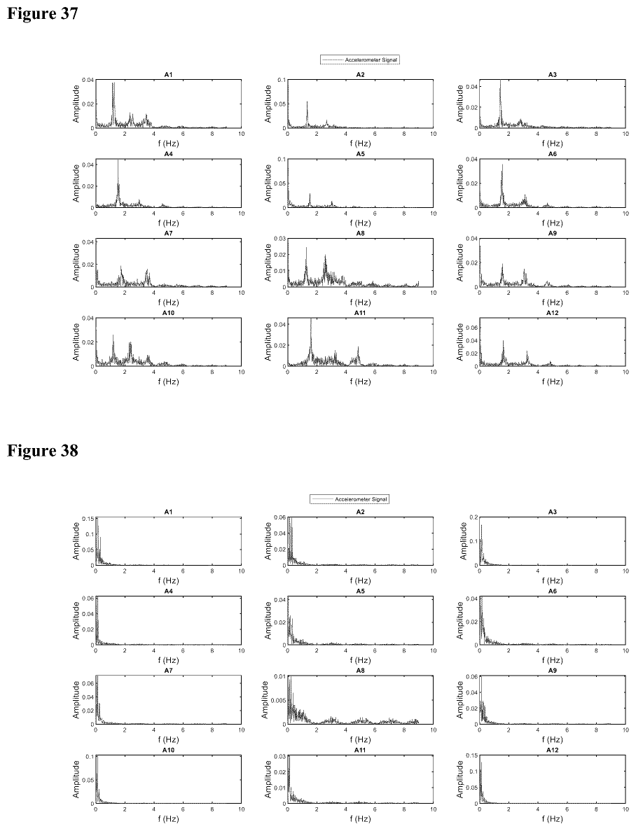

Shallow Breathing or hyperventilation is an abnormality in the breathing that may occur due to lung disease, and a study finding showed that before heart failure shallow breathing may worsen. The person may breath faster compared to normal breathing to meet respiratory needs. The participants in this case were lying on the bed and the IMU sensor was attached to the twelve (12) locations. FIG. 37 shows FFT of the data obtained for the twelve (12) different locations of the body. As per the reference signal shown in FIGS. 30 and 31, the frequency comes out to be 1.5 Hz. In FIG. 37, all the three locations have three major frequency components, the prominent one ranges from 1.2-1.7 Hz with best locations coming near as A5, A6, A8, A9, All and A4 with frequency as 1.2-1.5 Hz, very similar to the reference signal that is 1.3 Hz.

### Yawning Breathing Pattern

The signals acquired for yawning went through signal processing phase, where noise in the signal was reduced using the filter sgolayfilt, which is MATLAB function for Savitzky-Golay filter. As per the reference signal, the yawning frequency was determined to be 0.13 Hz. All the locations were able to detect the breathing with values ranging from 0.11 Hz to 0.14 Hz and close to the reference signal. However, the locations A1, A3, A4, A5, A6, A7, A8, A10, A11 and A12 were preferable.

### Coughing Breathing Pattern

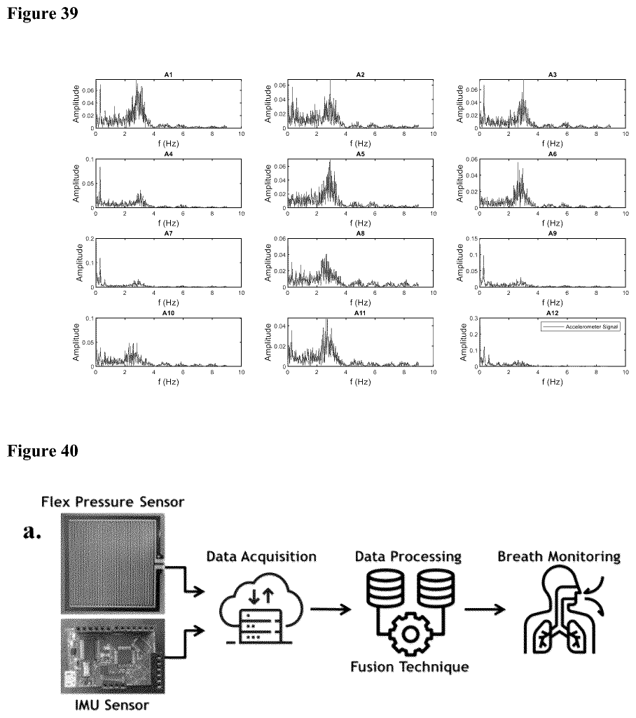

Episodes of coughing is helpful in detecting any lung related issue because it may be among first responses for any foreign element in the airways. FIG. 39 shows responses from different locations for coughing. The filtered signal was converted to frequency domain using MATLAB's predefine function, and the twelve (12) location plots are shown in FIG. 39, which showed the frequency ranged from 0.2-2.5 Hz. The locations A1, A2, A3, A5, A6, A8 and A11 seemed to detect stronger signal. Compared to the spirometer result from FIGS. 30 and 31 which various between 0.2 Hz to 2.8 Hz.

### Validation With Other Participants

The frequency domain analysis was conductted with four locations A5, A6, A8 and A9 as shown in FIG. 28, and which were selected based on the understanding developed above. For each location, all four breathing patterns were repeated and the data were compared to the reference signal. Similarity percentage between the accelerometer data and the reference signal is shown in Table 3 below (showing percentage match of breathing sensor with the spirometer results), with a view to validate the locations with other participants of the same age group:

TABLE 3

| | Breathing | | | | |
|---|---|---|---|---|---|
| Participants(K) | Patterns | A5 | A6 | A8 | A9 |
| K = 2 | Normal | 98 | 98 | 0 | 98 |
| | Shallow | 0 | 94 | 95 | 96 |
| | Yawning | 97 | 98 | 86 | 83 |
| | Coughing | 98 | 97 | 0 | 90 |
| | Normal | 87 | 91 | 0 | 87 |
| K = 3 | Shallow | 90 | 91 | 80 | 82 |
| | Yawning | 99 | 99 | 98 | 98 |
| | Coughing | 92 | 96 | 95 | 0 |
| | Normal | 96 | 98 | 98 | 96 |
| K = 4 | Shallow | 93 | 99 | 99 | 94 |
| | Yawning | 99 | 97 | 98 | 95 |
| | Coughing | 97 | 95 | 95 | 97 |
| | Normal | 98 | 99 | 98 | 98 |
| K = 5 | Shallow | 95 | 95 | 95 | 96 |
| | Yawning | 100 | 100 | 99 | 100 |
| | Coughing | 95 | 85 | 97 | 96 |
| | Normal | 95 | 98 | 88 | 96 |
| K = 6 | Shallow | 94 | 90 | 92 | 80 |
| | Yawning | 100 | 93 | 94 | 100 |
| | Coughing | 98 | 96 | 97 | 98 |

Table 3 shows different locations and their average as 91, 95, 80, and 89. A6 locations had the highest match. Upon a closer look, the locations A8 and A9 appeared more suitable for coughing breathing pattern. The cell marked as 0 are discarded values due to higher errors. The participants were asked to repeat the breathing patterns, as variations at different locations may be affected by other factors like the human thermal plume, age and physiological factors.

### Conclusion

The breathing sensor was able to detect all different breathing patterns for all twelve (12) locations. To further narrow down the locations the time domain analysis, frequency domain analysis and the biological factors were considered, which indicated that the location A6 may be preferable detecting different breathing patterns on healthy individuals. The locations A9 and A8 performed well specifically for coughing breathing patterns.

For contact based breathing sensors, the ability to detect breathing correctly is one key for determining different breathing patterns. Reliable and consistent sensor performance may depend on sensor placement due to differences in the movement of the chest muscles. Hence, this study may provide some understanding of placement criteria and experimental results for healthy individuals in the age group of 22-30.

In yet another study, a breath monitoring system was developed to operate on noninvasive measurements. The system primarily comprises three subsystems: the sensor subsystem, the microcontroller subsystem, and the data acquisition and analysis subsystem. Throughout the breathing process, the periodic motion of the thoracic and abdominal areas can be captured by different sensors, such as IMU sensors and flex sensors. Inhalation involves the diaphragm contracting, pushing the abdominal organs downward, and consequently reducing intrathoracic pressure. Conversely, during exhalation, as the diaphragm relaxes, the chest and abdomen return to their resting positions, leading to a reduction in lung volume.

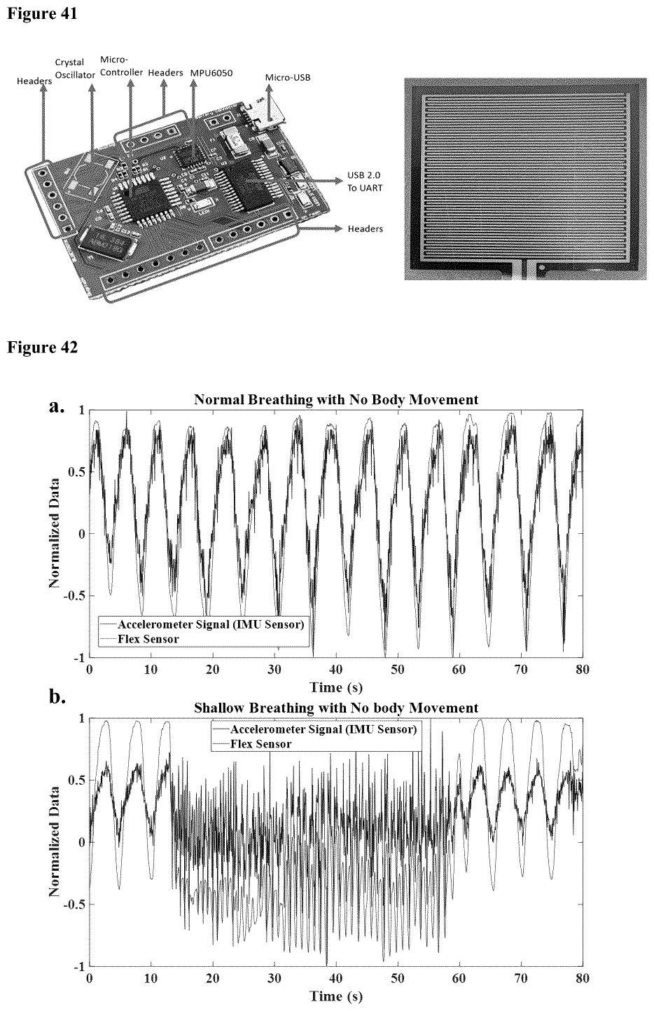

The procedure for data collection and analysis, along with its corresponding experimental setup, is illustrated in FIG. 40. In the setup, IMU and flex sensors were used to detect and monitor breathing, and a spirometry is applied to obtain the actual breathing signal to evaluate the performance of the approach. Six young (mean age: 23.2 years, SD: 2.4) adults (3 male) were recruited for testing the system. The study procedure was approved by the University of Windsor's research ethics board (REB #22-166), and was in accordance with the Declaration of Helsinki, except for registration in a clinical database. The IMU sensor, flex sensor and spirometer are explained below.

FIG. 41 shows the hardware used for the breathing sensor. The breathing sensor system was made with a four-layer PCB having two main components, the microcontroller (Atmega328P) and the IMU sensor (MPU6050), which can measure 3-axis acceleration and 3-axis rotation motion. The board was synchronized with a 16.384 MHz oscillator. The Atmega328P includes 2 KB SRAM, 32 KB Flash and 1 KB EEPROM. The EEPROM is a nonvolatile memory, meaning that the data remain the same even if the power is off. Hence, once the board is programmed, it does not need to be programmed again. The MPU6050 communicates with the microcontroller via the I2C protocol. The MPU6050 detects inertial movement. Flex pressure sensor data were collected via one of the analog pins on the board.

The flex sensor was connected to the analog pin present in the header. The headers represent various analog and digital pins obtained from the microcontroller. The IMU sensor was connected to the microcontroller via electrical traces on the board. The Micro-USB was used to communicate with a laptop on two occasions. First, the breathing sensor was programmed to read all the acceleration and rotation data using an Arduino IDE. The programming was performed only once, as the board has the memory to save the code. Second, the breathing sensor was used to read and plot the data continuously via MATLAB-based code. At the end of each session, the data were saved in an Excel file. This event was the main data acquisition event and was repeated each time during the experiment.

For the spirometry setup, the participants were instrumented with a mouthpiece and nose clip. The mouthpiece was connected to a calibrated respiratory flow head and high-precision spirometer (ADInstruments®, Colorado Springs, USA). The respiratory rates and volumes were determined in real time using PowerLab and LabChart Pro software (ADInstruments®).

### Sensor Data Fusion

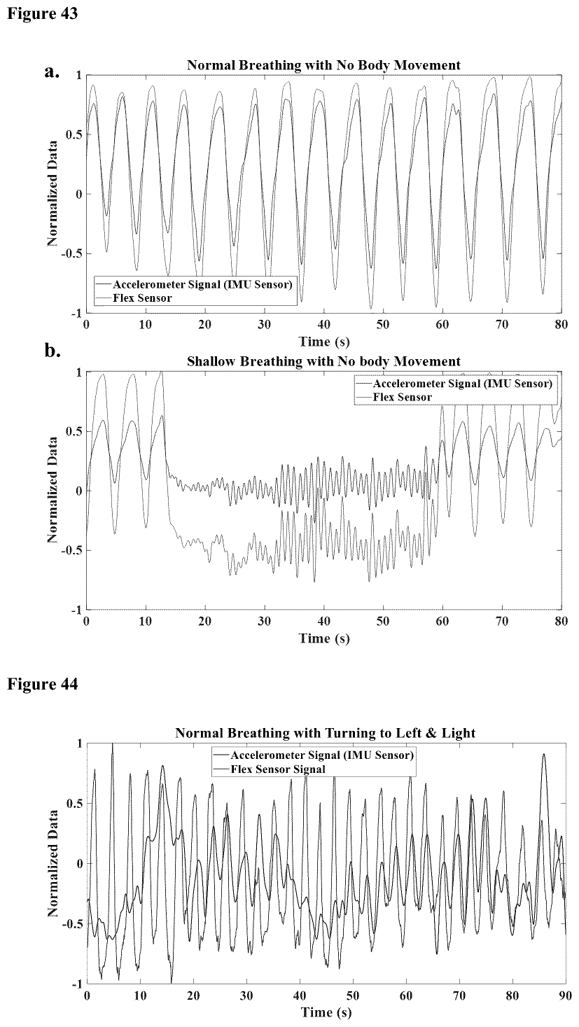

The main challenge addressed was to isolate breathing-related motion from body movement through sensor data fusion. To understand significance of sensor data fusion and determine an appropriate method for performing the fusion, performance of the IMU and flex sensors was evaluated individually. For this purpose, the ability of both sensors to measure breathing signals was studied first. Flex sensors can capture muscle stretching and relaxation at the location to which they are attached. Meanwhile, IMU sensors can capture various body movements such as rolling, bending, walking, and running. To detect abdominal movement using the IMU sensor, accelerometer data in the normal direction were utilized. FIG. 42 shows the accelerometer data in the normal direction and the flex sensor data during normal (FIG. 42(a)) and shallow breathing (FIG. 42(b)). It is important to note that there was no body movement during these breathing tests. After applying a low-pass filter to address the high noise-to-signal ratio of the accelerometer data, the filtered accelerometer signals for normal and shallow breathing are depicted in FIG. 43. After filtering, it was demonstrated that both signals provided consistent information, and monitoring breathing was possible based on the measurements of each sensor in the absence of body motion.

To assess the performance of these sensor signals in the presence of body movement, a normal breathing test was conducted while the participant's body was gently turning to the left and right. As depicted in FIG. 44, the results highlight the significant impact of body motion on the accelerometer data, whereas the flex sensor data exhibit only minor variation. Consequently, the flex sensor appeared more robust to body motion than the IMU sensor. It was evident that if the accelerometer and flex sensor data are fused, the fusion does not yield additional information compared to using only the flex sensor data in the presence of body motion. This observation led to the question of whether relying solely on a flex sensor could be adequate for breath monitoring.

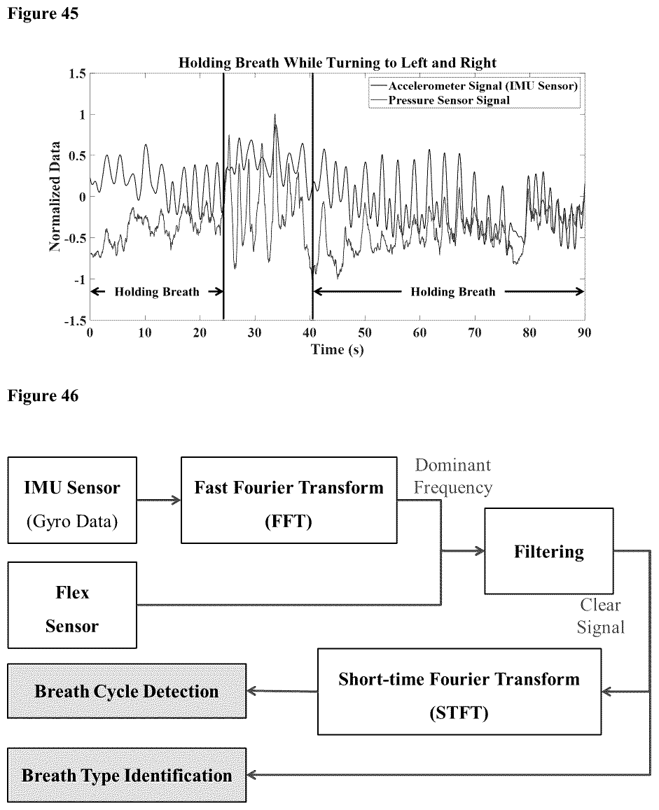

To address this issue, a test involving breath holding during body motion was conducted. FIG. 45 shows a breathing test that includes two intervals of breath-holding while the participant turned to the left and right. During these intervals, it was anticipated that the flex sensor data should have remained nearly constant. However, due to muscle motion during turns to the right and left, the flex sensor data exhibited cyclic behavior corresponding to the body's motion. This cyclical pattern introduced the risk of false alarms or readings when detecting breathing. Consequently, relying solely on a flex sensor may not appear sufficient. Therefore, it was proposed to integrate flex sensor data with other sensory information.

It has been appreciated it may be possible that the previously mentioned false alarms or readings may stem from the motion of abdominal muscles, including contraction and relaxation, induced by the body's rotational movement. Consequently, it may be preferable to extract and incorporate information about the body's rotational motion to enhance the performance of flex sensor data.

### IMU and Flex Sensor Data Fusion Technique

A fusion technique was developed based on excluding information regarding body rotation from flex sensor data to achieve a signal that more purely includes breath-related information. An overview of the method is depicted in FIG. 46. To capture information about the body's rotational motion along three axes (x, y, z), the gyro signals from the IMU sensor were utilized. Fast Fourier transformation (FFT) transforms the signal from the time domain to the frequency domain, providing a spectrum that illustrates the frequency components present in that signal. Employing the FFT allows identification of frequencies associated with the body's rotational motion. Once the dominant frequencies of body rotational motion are determined, they are removed from the flex sensor data through filtering. As a result, the filtered flex sensor data exclusively contain information related to breathing, making them suitable for breath monitoring.

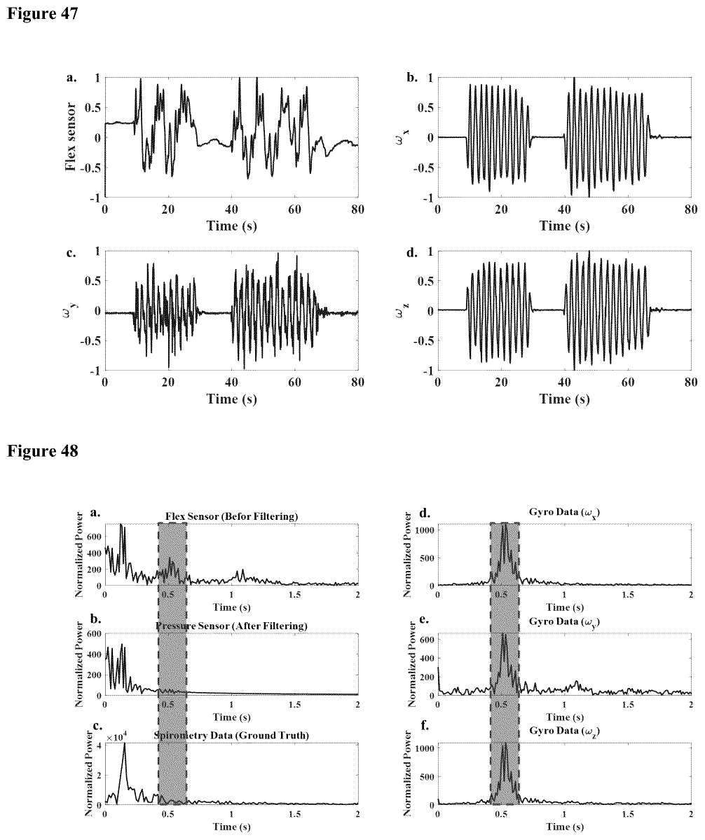

To assess effectiveness, an experimental test involving deep breathing with motion was conducted. FIG. 47 shows the flex sensor data and gyro signals along the three axes, and the fast Fourier transform (FFT) of these signals is shown in FIG. 48. Based on the FFT of the gyro data, the region of dominant frequencies corresponding to the body motion is identified and highlighted in this figure. For this specific experimental test, the dominant frequencies fall within the range of 0.45 to 0.6 Hz. The FFT analysis of the flex sensor data also revealed the presence of these frequencies (FIG. 48(a)), but they were not dominant in the spirometer data (FIG. 48(c)). By applying the technique, these frequencies were removed from the flex sensor data. As shown in FIG. 48(b), the FFT of the filtered flex sensor data closely resembles the FFT of the spirometer data, indicating the absence of frequencies corresponding to body motion in this range. Obtaining a signal that contains only breath-related information allows determination of the number of breaths or the breathing rate and to determine the breathing type.

### Counting the Number of Breath Cycles or Calculating the Breathing Rate

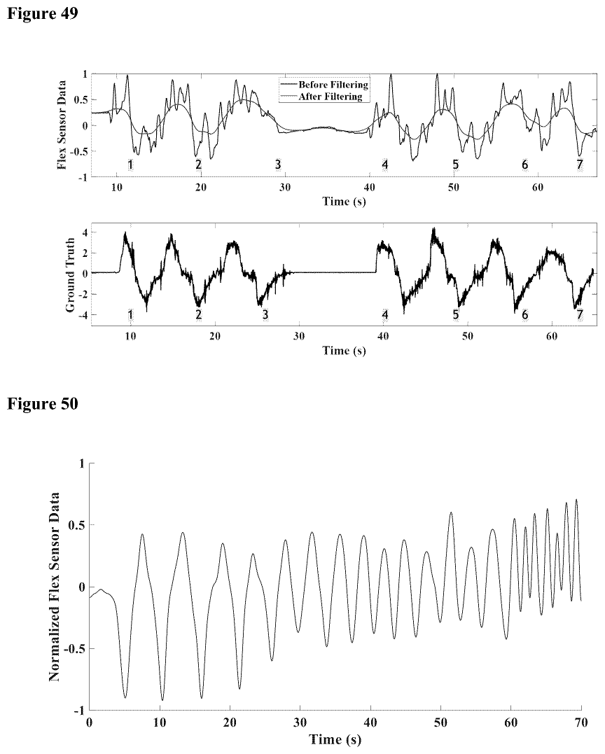

Flex sensor data before and after filtering, and spirometer signal, serving as the reference, are illustrated in FIG. 49. Before applying the technique, the flex sensor data had 31 peaks, indicating 31 breath cycles. However, after applying the developed fusion technique, the number of breath cycles extracted from the filtered flex sensor data is 7, which is consistent with the count obtained using the reference. This showed that in the test, body motion led to a false alarm rate of more than 300% in counting the breath cycles when the technique is not applied. The results highlight effectiveness of the technique to avoid or reduce counting cycles corresponding to body motion as breath cycles.

It has been appreciated that sensor data fusion may be applied with signals from an accelerometer or with signals from both the gyroscope sensor and the accelerometer to filter the flex sensor data to remove false readings, which may result from rotational movements, as well as linear or substantially linear movements.

### Identifying the Breath Type

The short-time Fourier transform (STFT) was employed to analyze and understand temporal frequency characteristics of a signal. It has been appreciated that in the realm of breathing analysis, the STFT may provide valuable resource. The procedure was initiated by segmenting the respiratory data into brief time intervals, with each window depicting a small segment of the total signal duration. The rationale behind selecting these short time intervals was to capture the dynamic variations in the breathing pattern as it evolves over time. Upon segmenting the signal, the fast Fourier transform (FFT) was applied to each window individually. This process allowed insights to be gained regarding the evolution of the frequency content of the breathing signal over time. To avoid overlooking vital information, overlapping windows were employed. This approach facilitated comprehensive coverage of the entire signal duration, more accurately capturing nuances and variations in the breathing pattern.

In the context of determining breathing types, the STFT facilitated a more thorough exploration of frequency variations over short intervals. This method permitted identification of specific patterns or characteristics associated with different breathing types. This analysis serves to differentiate between various breathing patterns and enhances the overall accuracy of breath monitoring systems.

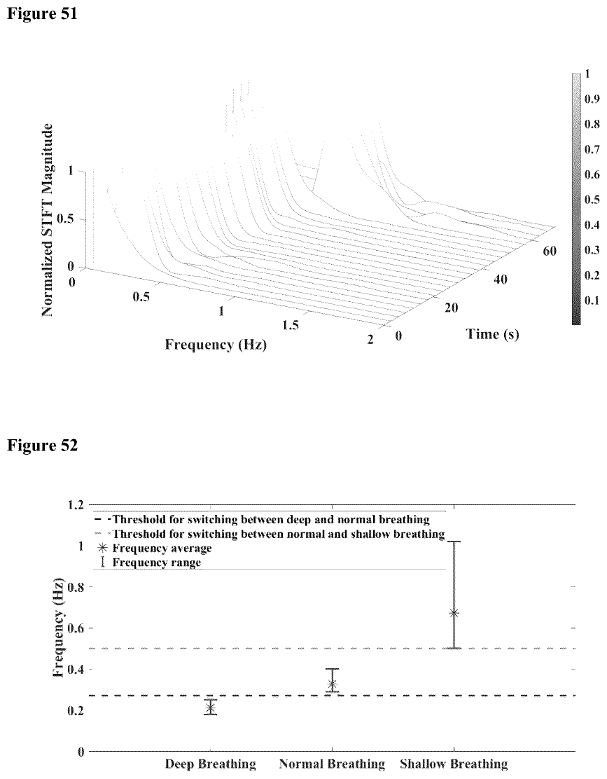

To assess how the technique may identify the type of breathing, a test involving deep, normal, and shallow breathing was considered. After applying the developed fusion technique and filtering non-respiratory information from the flex sensor data depicted in FIG. 50, the signal was segmented into 6-second time intervals with a 3-second overlap. Subsequently, the FFT was applied to each time interval to extract information concerning the frequencies present within that interval. FIG. 51 displays the STFTs of the experimental tests and reveals the dominant frequencies within each time interval. The most dominant frequency in each interval corresponded to the maximum magnitude of the FFT within that interval. It has been recognized that by determining thresholds as conditions for switching between breath types and utilizing the dominant frequency in each time window, breath types may be classified.

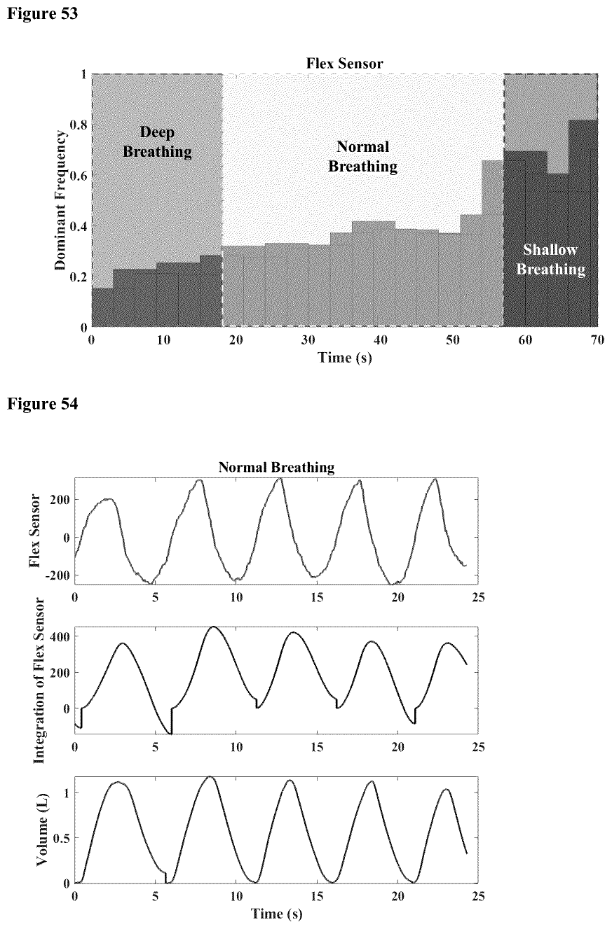

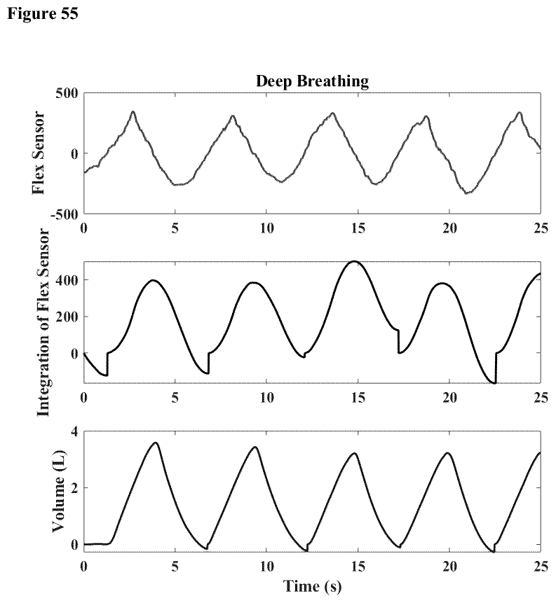

To determine distinct thresholds for breath type identification, experimental tests were conducted. Each participant was instructed to perform deep, normal, and shallow breathing. FIG. 52 illustrates the ranges of dominant frequencies observed during these tests. Based on the results, the threshold between the two types of breathing was calculated by averaging the mean frequency of each breathing type. Based on FIG. 51, the most dominant frequency of the experimental test within each time interval is shown in FIG. 53. Using defined thresholds, it was possible to determine the types of breathing present in the test and determine the duration of each type of breathing. Based on the classification results, the number of breath cycles for each type may be determined. Table 4 below compares these results with the actual number of breath cycles the participant was asked to take.

TABLE 4
Number of breath cycles in each breath type

| | Number of breath cycles | |
|---|---|---|
| | determined by developed | Actual Number |
| | approach | of breath cycles |
| Deep Breathing | >2 | 4 |
| Normal Breathing | >11 | 11 |
| Shallow Breathing | 7 | 6 |
| All types of | 20 | 20 |
| Breathing | | |

The results showed that the technique may permit determination of the number of cycles in each breath type. Considering 6-second time intervals with a 3-second overlap may cause errors of 3 seconds in the identification of breath types. Another source of slight mismatches between the calculated results and the actual ones may arise from the identification of breath types during the transition from one breath type to another, which can be complicated to classify.

These thresholds may vary among individuals and be influenced by factors such as sex, age, weight, and height, such that personalization may be preferable. By instructing individuals to engage in these specific breathing types and subsequently calibrating or adjusting the device based on their unique thresholds, the sensor device may be more specific to each person's individual characteristics.

Similarly, as seen in FIGS. 53 and 54, the filtered time domain flex sensor data can be used to determine breathing or tidal volume by, for example, integrating and normalizing the sensor data against a standard signal to volume calibration curve previously obtained for each individual.

### Conclusions

The foregoing demonstrates an approach for monitoring breathing rates through the integration of flex and inertial measurement unit (IMU) sensor data. The fusion technique may permit reduction or elimination of non-respiratory information from flex and IMU data to extract breath-related information and allow improved sensitivity of flex sensors to motion-related artifacts. The experimental results showed that by applying the fusion technique, non-respiratory information may be filtered from the flex and IMU data, resulting in a signal containing more accurate information about breathing. The results demonstrated that based on the obtained signal, it was possible to more accurately count breath cycles. Moreover, by segmenting the obtained signal into short time intervals and analyzing the dominant frequency in each time interval, different breath types were identified.

While the invention has been described with reference to preferred embodiments, the invention is not or intended by the applicant to be so limited. A person skilled in the art would readily recognize and incorporate various modifications, additional elements and/or different combinations of the described components consistent with the scope of the invention as described herein.

We claim:

1. A method for determining a breathing pattern, the method comprising:
placing a motion sensor and a flex sensor proximate to a diaphragm of a subject;
obtaining time domain signals or data from both said sensors over a time period;
subjecting the time domain signals or data from the motion sensor to Fast Fourier transformation to obtain frequency domain signals or data, and determining from the frequency domain signals or data one or more frequencies associated with body motion of the subject substantially unrelated to breathing; and
filtering the time domain signals or data from the flex sensor to remove information related to the body motion therefrom to obtain filtered time domain signals or data.

2. The method of claim 1, wherein the one or more frequencies associated with the body motion of the subject at least partially form a peak in the frequency domain signals or data.

3. The method of claim 1, wherein the breathing pattern comprises a breathing frequency, the method further comprising determining the breathing frequency from the filtered time domain signals or data.

4. The method of claim 1, wherein the breathing pattern comprises a breathing type, the method further comprising: subjecting the filtered time domain signals or data to short-time Fourier transformation with a plurality of shorter time segments over the time period to obtain a spectrogram; identifying a dominant frequency peak for each said segment in the spectrogram; and determining the breathing type for each said segment based on the dominant frequency peak.

5. The method of claim 4, wherein the breathing type comprises slow breathing, normal breathing and fast breathing, the method further comprising determining respective breathing frequency ranges of the slow breathing, normal breathing and fast breathing, wherein said determining the breathing type comprises determining the breathing type for each said segment based on proximity or overlap of the dominant frequency peak to the respective breathing frequency ranges of the slow breathing, normal breathing and fast breathing.

6. The method of claim 4, wherein each said shorter time segment has a duration between 1 second and 10 seconds, and each said shorter time segment overlaps with at least one other said shorter time segment for an overlap duration between 0.5 second and 5 seconds.

7. The method of claim 1, wherein one or both of the motion sensor and the pressure sensor are for placement between ribs 7 and 9 on the left side of the subject.

8. The method of claim 1, wherein the method further comprises subjecting the time domain signals or data to one or both of a Savitzky-Golay filter and a low pass filter.

9. The method of claim 1, wherein the motion sensor comprises an inertial measurement unit having an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

10. The method of claim 1, wherein the motion sensor comprises one or both of an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

11. The method of claim 1, wherein the flex sensor comprises a resistive or capacitive flex sensor.

12. A system for determining a breathing pattern, the system comprising a motion sensor, a flex sensor and an information processing unit, wherein the sensors are for placement proximate to a diaphragm of a subject, and the information processing unit is configured to:
obtain time domain signals or data from both said sensors over a time period;
subjecting the time domain signals or data from the motion sensor to Fast Fourier transformation to obtain frequency domain signals or data, and determining from the frequency domain signals or data one or more frequencies associated with body motion of the subject substantially unrelated to breathing; and
filtering the time domain signals or data from the flex sensor to remove information related to the body motion therefrom to obtain filtered time domain signals or data.

13. The system of claim 12, wherein the one or more frequencies associated with the body motion of the subject at least partially form a peak in the frequency domain signals or data.

14. The system of claim 12, wherein the breathing pattern comprises a breathing frequency, the information processing unit being further configured to determine the breathing frequency from the filtered time domain signals or data.

15. The system of claim 12, wherein the breathing pattern comprises a breathing type, the information processing unit being further configured to: subject the filtered time domain signals or data to short-time Fourier transformation with a plurality of shorter time segments over the time period to obtain a spectrogram; identify a dominant frequency peak for each said segment in the spectrogram; and determine the breathing type for each said segment based on the dominant frequency peak.

16. The system of claim 15, wherein the breathing type comprises slow breathing, normal breathing and fast breathing, the information processing unit being configured to determine the breathing type for each said segment based on proximity or overlap of the dominant frequency peak to respective breathing frequency ranges of the slow breathing, normal breathing and fast breathing, wherein the respective breathing frequency ranges of the slow breathing, normal breathing and fast breathing are predetermined from the subject.

17. The system of claim 15, wherein each said shorter time segment has a duration between 1 second and 10 seconds, and each said shorter time segment overlaps with at least one other said shorter time segment for an overlap duration between 0.5 second and 5 seconds.

18. The system of claim 12, wherein one or both of the motion sensor and the flex sensor are for placement between ribs 7 and 9 on the left side of the subject.

19. The system of claim 12, wherein the motion sensor and the flex sensor are for placement on a patch to be applied to skin of the subject.

20. The system of claim 12, wherein the information processing unit is further configured to subject the time domain signals or data to one or both of a Savitzky-Golay filter and a low pass filter.

21. The system of claim 12, wherein the motion sensor comprises an inertial measurement unit having an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

22. The system of claim 12, wherein the motion sensor comprises one or both of an accelerometer and a gyroscope sensor, said subjecting the time domain signals or data from the motion sensor to Fast Fourier transformation comprising subjecting the time domain signals or data from one or both of the accelerometer and the gyroscope sensor to Fast Fourier transformation to obtain the frequency domain signals or data.

23. The system of claim 12, wherein the flex sensor comprises a resistive or capacitive flex sensor.

24. A patch system for determining a breathing pattern, the system comprising a motion sensor and a flex sensor, both said sensors being placed on a patch for application to skin of a subject between ribs 7 and 9 on the left side of the subject.

25. A method for determining a respiration events, the method comprising:
placing an inertial measurement unit (IMU) positioned on the skin proximate to the diaphragmatic area of the subject to detect breathing-induced acceleration motion and non-breathing related body motion and generate representing time domain data of the signal from the subject;
placing a flex pressure sensor positioned on the skin proximate to the diaphragmatic area of the subject to detect breathing-induced muscle stretch and generate representing time domain data of the signal from the subject;
obtaining time domain signals or data from both said inertial measurement unit and flex sensors over a time period;
subjecting the time domain signals or data from the inertial measurement unit (consisting of tri-axis accelerometer and tri-axis gyroscope) sensor to Fast Fourier transformation to obtain frequency domain signals or data, and determining from the frequency domain signals or data one or more frequencies associated with non-breathing related body motion of the subject; and
filtering the time domain signals or data from the pressure sensor to remove information related to the body motion therefrom to obtain filtered time domain signals or data.

26. A system for determining a breathing pattern, the system comprising a gyroscope and accelerometer sensor, a pressure sensor and an information processing unit, wherein the sensors are for placement proximate to a diaphragm of a subject, and the information processing unit is configured to:
obtain time domain signals or data from both said sensors over a time period;
subjecting the time domain signals or data from the accelerometer sensor to Fast Fourier transformation to obtain frequency domain signals or data, and determining from the frequency domain signals or data one or more frequencies associated with body motion of the subject; and
filtering the time domain signals or data from the pressure sensor to remove information related to the body motion therefrom to obtain filtered time domain signals or data.
